(19) United States
(12) Patent Application Publication
(10) Pub. No.: US 2019/0076114 A1
(43) Pub. Date: Mar. 14, 2019

(54) SYSTEM AND METHOD FOR ENERGY TRANSFER

(71) Applicant: QUALCOMM Incorporated, San Diego, CA (US)

(72) Inventors: Firas Sammoura, Dublin, CA (US); David William Burns, San Jose, CA (US); Ravindra Vaman Shenoy, Dublin, CA (US)

(21) Appl. No.: 15/703,746

(22) Filed: Sep. 13, 2017

(51) Int. Cl. A61B 8/12; A61B 6/00; A61B 8/08; G01S 15/89; B06B 1/06; B06B 1/02

(52) U.S. Cl. CPC A61B 8/12; A61B 6/4057; A61B 8/0808; A61B 2562/028; B06B 1/064; B06B 1/0292; G01S 15/895; G01S 15/8918

(57) ABSTRACT

An apparatus may include an array of piezoelectric micromachined ultrasonic transducers (PMUTs) and a control system configured to communicate with the array of PMUTs. The control system may be configured to determine a target location within a human body and to control the array of PMUTs to focus ultrasonic waves at the target location.

### TECHNICAL FIELD

This disclosure relates to implantable medical devices (IMDs) and more specifically to methods and devices for providing power to IMDs.

### DESCRIPTION OF THE RELATED TECHNOLOGY

Implanted medical devices generally require a continuous or quasi-continuous source of power. For example, power is needed for electronic components in neural modulation implants, insulin monitors and delivery systems, pacemakers, cochlear implants, neurostimulation devices for epilepsy stabilization or for Parkinson's treatments, etc. Currently, batteries are used to power implantable devices. However, batteries have a limited lifetime. The surgery required for replacing a battery in a deeply-implanted medical device may be non-trivial. Recently, radio frequency (RF)-based power transmission methods have been developed for recharging a battery of an implanted device. However, the Food and Drug Administration (FDA) mandates an RF intensity limit of only 0.1 mW/mm², in order to avoid possible tissue damage. Moreover, RF energy is significantly attenuated by human tissue.

### SUMMARY

The systems, methods and devices of the disclosure each have several innovative aspects, no single one of which is solely responsible for the desirable attributes disclosed herein. One innovative aspect of the subject matter described in this disclosure can be implemented in an apparatus that includes an array of piezoelectric micromachined ultrasonic transducers (PMUTs) and a control system. The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof.

The control system may be configured to communicate with the array of PMUTs. In some examples, the control system may be configured to determine a target location within a human body and to control the array of PMUTs to focus ultrasonic waves at the target location.

According to some examples, one or more PMUTs in the array of PMUTs may have a curved surface when in a static position. According to some implementations, the apparatus may include a substrate on which at least a portion of the array of PMUTs is disposed. In some examples, the substrate may have a curvature that is configured to focus ultrasonic energy emitted by the PMUTs that are disposed on the substrate.

In some examples, one or more PMUTs in the array of PMUTs may include a piezoelectric layer, a first electrode on a first side of the piezoelectric layer and a second electrode on a second side of the piezoelectric layer. In some such examples, one or more of the PMUTs does not include a deformable structural layer proximate the first side or the second side of the piezoelectric layer. In some such examples, the piezoelectric layer, the first electrode and the second electrode may reside on a support structure. At least a portion of a support structure area may extend beyond an area of the piezoelectric layer. In some implementations, the first electrode may be a center electrode and/or a ring electrode.

According to some implementations, a first portion of the piezoelectric layer may span a cavity region and a second portion of the piezoelectric layer may be mechanically coupled to a support structure adjacent the cavity region. The second portion of the piezoelectric layer and the support structure may combine to produce a mechanical moment on the first portion of the piezoelectric layer when a transmitter excitation signal is applied to one of the first electrode or the second electrode. The produced mechanical moment may result in a transverse deflection of the one or more PMUTs in the array of PMUTs. In some such examples, the first electrode and the second electrode span the entire cavity region. In some implementations, one or more PMUTs in the array of PMUTs also may include a deformable structural layer that spans the cavity region.

In some examples, controlling the array of PMUTs to focus ultrasonic energy at the target location may involve at least one of changing a curvature of a substrate on which the array of PMUTs resides, performing a beam steering process, or changing an orientation of one or more PMUT diaphragms.

According to some examples, one or more PMUTs of the array of PMUTs may be configured to detect received ultrasonic waves. Determining the target location may be based, at least in part, on received ultrasonic waves that are reflected from or transmitted from the target location.

In some such examples, the control system may be configured to control the array of PMUTs to scan a region inside the human body with transmitted ultrasonic waves. According to some such examples, the array of PMUTs may include one or more PMUTs configured to transmit ultrasonic waves. For example, the one or more PMUTs configured to transmit ultrasonic waves may include a piezoelectric material having a higher piezoelectric coefficient, a higher dielectric constant and/or a smaller thickness relative to the piezoelectric material of the one or more PMUTs configured to detect received ultrasonic waves.

According to some implementations, the control system may be configured to control a power level and/or a focal area of at least a portion of the array of PMUTs according to one or more signals received from a device implanted within the human body.

In some examples, the target location may correspond with at least a portion of a device implanted within the human body. For example, the target location may correspond with a second array of PMUTs of the device implanted within the human body. The control system may be configured to control the array of PMUTs for ultrasonic energy transmission to the device implanted within the human body.

According to some examples, one or more PMUTs in the array of PMUTs may include at least one edge electrode. The edge electrode may be configured to orient a PMUT diaphragm in the array of PMUTs towards the target location.

Other innovative aspects of the subject matter described in this disclosure may be implemented in a method of controlling an array of PMUTs. The method may involve determining a target location within a human body based, at least in part, on received ultrasonic waves that are reflected from or transmitted from the target location. The received ultrasonic waves may be received by one or more PMUTs of the array of PMUTs that are configured for detecting received ultrasonic waves. The method may involve controlling the array of PMUTs to focus ultrasonic waves at the target location.

In some examples, the method may involve controlling the array of PMUTs to scan a region inside the human body with transmitted ultrasonic waves. The method may involve controlling the array of PMUTs to transmit ultrasonic energy to a device implanted within the human body. In some instances, the method may involve controlling a power level and/or a focal area of at least a portion of the array of PMUTs according to one or more signals received from a device implanted within the human body.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in a non-transitory medium having software stored thereon. For example, the software may include instructions for causing a processor to determine a target location within a human body based, at least in part, on received ultrasonic waves that are reflected from or transmitted from the target location. The received ultrasonic waves may be received by one or more PMUTs of an array of PMUTs configured for detecting received ultrasonic waves. The software may, in some examples, include instructions for causing the processor to control the array of PMUTs to focus ultrasonic waves at the target location.

In some implementations, the software may include instructions for causing a processor to: control the array of PMUTs to scan a region inside the human body with transmitted ultrasonic waves. In some examples, the software may include instructions for causing a processor to: control the array of PMUTs to transmit ultrasonic energy to a device implanted within the human body. According to some implementations, the software may include instructions for causing a processor to: control a power level and/or a focal area of at least a portion of the array of PMUTs according to one or more signals received from a device implanted within the human body.

Other features, aspects, and advantages will become apparent from a review of the disclosure. Note that the relative dimensions of the drawings and other diagrams of this disclosure may not be drawn to scale. The sizes, thicknesses, arrangements, materials, etc., shown and described in this disclosure are made only by way of example and should not be construed as limiting.

### BRIEF DESCRIPTION OF THE DRAWINGS

FIG. 1 is a block diagram that shows example components of an apparatus according to some implementations.

FIG. 2 is a flow diagram that provides example blocks of some methods disclosed herein.

FIG. 3 shows one example of an array of PMUTs residing on a curved substrate.

FIG. 4A shows an example of a PMUT element that includes a deformable structural layer that is separate from a piezoelectric layer.

FIG. 4B shows an example of a CMUT element that includes a deformable structural layer without a piezoelectric layer.

FIGS. 4C-4R show alternative examples of PMUTs that include a deformable structural layer that is separate from a piezoelectric layer.

FIGS. 5A and 5B show a top view and a cross-sectional view, respectively, of one example of a PMUT having a curved surface when in a rest position.

FIGS. 5C-5R show alternative examples of PMUTs that do not include a deformable structural layer that is separate from a piezoelectric layer.

FIG. 6A shows an example of a PMUT array including PMUTs that are configured for detecting received ultrasonic energy.

FIG. 6B shows another example of a PMUT array including PMUTs that are configured for detecting received ultrasonic energy.

FIGS. 6C and 6D show a top view and a cross-sectional view, respectively, of a portion of the apparatus shown in FIG. 6B.

FIGS. 6E and 6F show a top view and a cross-sectional view, respectively, of an example of an implementation of a PMUT array configured for detecting, receiving and transmitting ultrasonic energy.

FIG. 6G shows a cross-sectional view of another implementation of a PMUT array configured for detecting, receiving and transmitting ultrasonic energy.

FIG. 6H shows a plan view of an implementation of a PMUT array configured for changing an orientation of one or more PMUT diaphragms in the PMUT array.

FIG. 6I shows a cross-sectional view through line 6i-6i′ of the implementation shown in FIG. 6H, with the PMUT array configured for changing the orientation of one or more PMUT diaphragms and for beam steering.

FIG. 6J shows a plan view of another implementation of a PMUT array configured for changing an orientation of one or more PMUT diaphragms in the PMUT array.

FIG. 6K shows a cross-sectional view through line 6k-6k′ of the implementation shown in FIG. 6J, with the PMUT array configured for changing the orientation of one or more PMUT diaphragms and for beam steering.

FIG. 7 shows an example of apparatus configured for determining a target location within a human body.

FIG. 8A is a flow diagram that outlines example blocks of a method of locating an implanted device within a human body and providing power to the implanted device.

FIG. 8B is a flow diagram that outlines example blocks of an alternative method of locating an implanted device within a human body and providing power to the implanted device.

FIG. 9 shows an example of apparatus configured to provide power to implantable devices used for deep brain stimulation (DBS).

FIG. 10 shows a more detailed example of one of the implantable devices of FIG. 9.

FIGS. 11A and 11B show examples of relatively rigid substrates that are connected by relatively flexible routing portions.

FIGS. 11C-11G show examples of PMUT arrays that are connected by relatively flexible routing portions.

Like reference numbers and designations in the various drawings indicate like elements.

### DETAILED DESCRIPTION

The following description is directed to certain implementations for the purposes of describing the innovative aspects of this disclosure. However, a person having ordinary skill in the art will readily recognize that the teachings herein may be applied in a multitude of different ways. The described implementations may be implemented in any device, apparatus, or system that includes a sensor system. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands and patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players (such as MP3 players), camcorders, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, steering wheels, camera view displays (such as the display of a rear view camera in a vehicle), electronic photographs, electronic billboards or signs, projectors, architectural structures, microwaves, refrigerators, stereo systems, cassette recorders or players, DVD players, CD players, VCRs, radios, portable memory chips, washers, dryers, washer/dryers, automated teller machines (ATMs), parking meters, packaging (such as in electromechanical systems (EMS) applications including microelectromechanical systems (MEMS) applications, as well as non-EMS applications), aesthetic structures (such as display of images on a piece of jewelry or clothing) and a variety of EMS devices. The teachings herein also may be used in applications such as, but not limited to, electronic switching devices, radio frequency filters, sensors, accelerometers, gyroscopes, motion-sensing devices, magnetometers, inertial components for consumer electronics, parts of consumer electronics products, varactors, liquid crystal devices, electrophoretic devices, drive schemes, manufacturing processes and electronic test equipment. Thus, the teachings are not intended to be limited to the implementations depicted solely in the Figures, but instead have wide applicability as will be readily apparent to one having ordinary skill in the art.

Some implementations disclosed herein may include an apparatus that includes an array of piezoelectric micromachined ultrasonic transducers (PMUTs, also referred to as “piezoelectric micromechanical ultrasonic transducers”) and a control system. The control system may be configured to determine a target location within a human body and to control the array of PMUTs to focus ultrasonic waves at the target location. The control system may be configured to control the array of PMUTs for ultrasonic energy transmission to a device implanted within the human body. In some examples, the control system may be configured to control the array of PMUTs to scan a region inside the human body with transmitted waves, such as transmitted ultrasonic waves. The apparatus may, in some examples, include a curved substrate on which at least a portion of the array of PMUTs is disposed. The substrate may have a curvature that is configured to focus ultrasonic energy emitted by the PMUTs that are disposed on the substrate. Alternatively, or additionally, one or more PMUTs in the array of PMUTs may have a curved surface when in a static position.

Particular implementations of the subject matter described in this disclosure can be implemented to realize one or more of the following potential advantages. Transmitting power to an implanted device acoustically has potential advantages as compared to the surgery required for replacing a battery in an implanted medical device. Transmitting power to an implanted device acoustically also has potential advantages as compared to transmitting power to an implanted device via RF energy. For example, the FDA intensity limit for energy applied to a human is 7.2 mW/mm² for ultrasound, as compared to 0.1 mW/mm² for RF energy. In addition, the energy attenuation for ultrasound caused by human tissue is approximately 1 dB/cm at 1 MHz, whereas the RF energy attenuation caused by human tissue is approximately 3 dB/cm at 2 GHz.

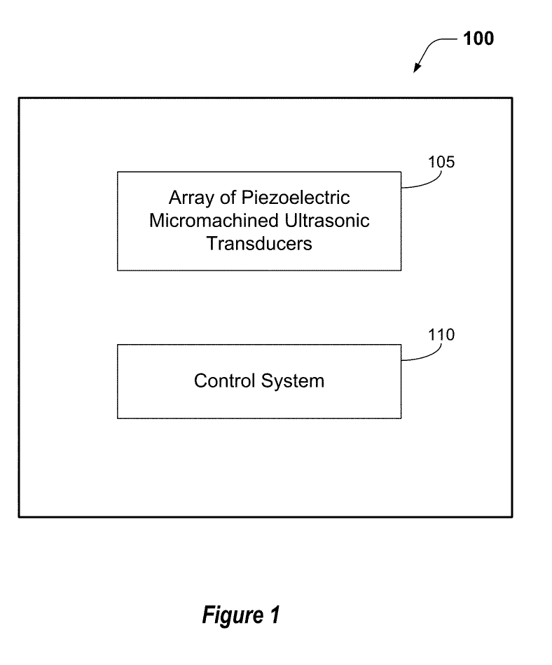

FIG. 1 is a block diagram that shows example components of an apparatus according to some implementations. In this example, the apparatus 100 includes an array of piezoelectric micromachined ultrasonic transducers (PMUTs) 105 and a control system 110 that is configured to communicate with the array of PMUTs 105. The control system 110 may be configured to communicate with the array of PMUTs 105 via wired communication and/or wireless communication. As used herein, the term “coupled to” includes being physically coupled for wired communication as well as being configured for wireless communication.

According to some implementations, the apparatus 100 may be, or may include, a wearable device. Various examples are disclosed herein. In some examples, the wearable device may be an implantable device.

In some implementations, at least a portion of the array of PMUTs 105 and/or the control system 110 may be included in more than one apparatus. In some examples, a second device (such as a mobile device) may include some or all of the control system 110, but may not include the array of PMUTs 105. However, the control system 110 may nonetheless be configured to communicate with the array of PMUTs 105.

The control system 110 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 110 also may include (and/or be configured for communication with) one or more memory devices, such as one or more random access memory (RAM) devices, read-only memory (ROM) devices and/or other types of non-transitory media. Accordingly, the apparatus 100 may have a memory system that includes one or more memory devices, though the memory system is not shown in FIG. 1.

The control system 110 may be capable of performing, at least in part, the methods disclosed herein. In some examples, the control system 110 may be capable of performing some or all of the methods described herein according to instructions (e.g., software) stored on non-transitory media. For example, the control system 110 may be configured for controlling the array of PMUTs 105 and/or for receiving and processing data from at least a portion of the array of PMUTs 105, e.g., as described below.

Various examples of PMUTs are disclosed herein. At least some PMUTs in the array of PMUTs 105 may be configured to transmit ultrasonic waves. According to some implementations, at least some PMUTs in the array of PMUTs 105 may be configured to receive ultrasonic waves. In some instances, one or more PMUTs that are configured to transmit ultrasonic waves may include a piezoelectric material having a higher piezoelectric coefficient, a higher dielectric constant and/or a smaller thickness relative to the piezoelectric material of one or more PMUTs configured to detect received ultrasonic waves.

In some examples, the array of PMUTs 105 may include one or more capacitive micromachined ultrasonic transducers (CMUTs), etc. As used herein, the term “PMUT” may be used in a broad sense that also includes CMUTs.

Although not expressly shown in FIG. 1, some implementations of the apparatus 100 may include an interface system. In some examples, the interface system may include a wireless interface system. In some such implementations, the apparatus 100 may be configured to receive, via the wireless interface system and/or via the array of PMUTs 105, signals from a device implanted within the human body and/or signals from a device outside the human body. In some instances, the apparatus 100 may be configured to receive instructions, via a wireless interface system, from a device outside the human body and to operate according to the received instructions. According to some such implementations, the apparatus 100 may be configured to transmit, via the wireless interface system, signals to a device implanted within the human body and/or signals to a device outside the human body.

Accordingly, in some implementations, the array of PMUTs 105 may be, or may be a part of, the interface system. In some such implementations, one or more PMUTs of the array of PMUTs may be configured to detect received ultrasonic waves. Determining the target location may be based, at least in part, on received ultrasonic waves that are reflected from or transmitted from the target location or from an area near the target location.

In some implementations, the interface system may include a user interface system, a network interface, an interface between the control system 110 and a memory system and/or an interface between the control system 110 and an external device interface (e.g., a port or an applications processor). In some examples, the interface system may include one or more wired or wireless interfaces between the control system 110 and one or more elements of the array of PMUTs 105. Accordingly, in some such implementations at least a portion of the array of PMUTs 105 and at least a portion of the control system 110 may reside in different devices. For example, at least a portion of the control system 110 may reside in a mobile device and one or more components of the array of PMUTs 105 may reside another device or in two or more other devices.

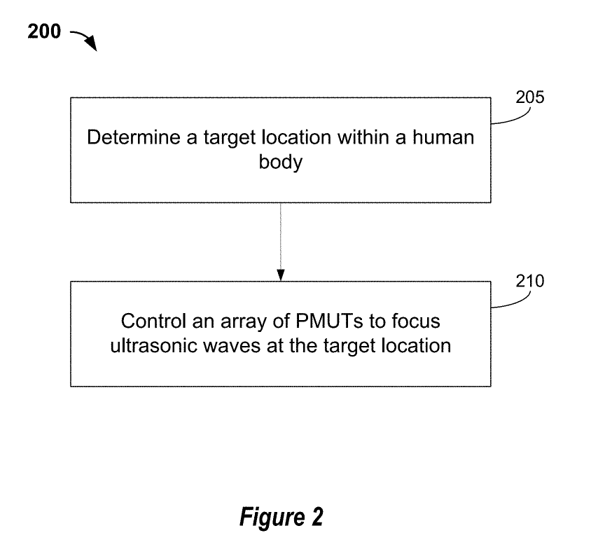

FIG. 2 is a flow diagram that provides example blocks of some methods disclosed herein. The blocks of FIG. 2 (and those of other flow diagrams provided herein) may, for example, be performed by the apparatus 100 of FIG. 1 or by a similar apparatus. As with other methods disclosed herein, the method outlined in FIG. 2 may include more or fewer blocks than indicated. Moreover, the operations of methods disclosed herein are not necessarily performed in the order indicated.

Here, block 205 involves determining a target location within a body, which is a human body in this example. In alternative implementations, block 205 may involve determining a target location within a body of another type of organism. In some examples, block 205 may involve a control system, such as the control system 110 of FIG. 1, controlling an array of PMUTs (such as the array of PMUTs 105) to scan a region inside the human body with transmitted waves. The transmitted waves may, for example, be transmitted ultrasonic waves. Determining the target location may be based, at least in part, on received ultrasonic waves that are reflected from or transmitted from the target location.

In some examples, block 205 may involve receiving signals from a device implanted within the human body. The signals may be electromagnetic signals, ultrasonic signals, etc., that are transmitted by a device implanted within the human body. According to alternative implementations, block 205 may involve detecting a magnetic field that corresponds with a target location inside the human body. In some such implementations, the apparatus 100 may include a magnetic field sensor, such as a MEMS-based magnetic field sensor.

In this example, block 210 involves controlling the array of PMUTs to focus ultrasonic waves at the target location. Block 210 may be performed in various ways, depending on the particular implementation. In some instances, the target location may correspond with at least a portion of a device implanted within the human body. The target location may correspond with a second array of PMUTs associated with the device implanted within the human body. Block 210 may, in some implementations, involve controlling the array of PMUTs for ultrasonic energy transmission to the device implanted within the human body.

However, in alternative implementations, the target location may correspond with a portion of the human body. Block 210 may, in some implementations, involve controlling the array of PMUTs for focused ultrasonic energy transmission to the portion of the human body. According to some such implementations, block 210 may involve controlling the array of PMUTs to provide focused ultrasonic energy for medical therapeutics.

In some examples, block 210 may involve a control system controlling the array of PMUTs to focus ultrasonic energy at the target location by changing a curvature of the substrate on which the array of PMUTs 105 resides, beam steering and/or changing an orientation of one or more PMUT diaphragms. However, according to some implementations, at least some aspects of focusing the acoustic energy may be accomplished without input from a control system. The apparatus 100 may, in some examples, include a curved substrate on which at least a portion of the array of PMUTs 105 is disposed. The substrate may have a curvature that is configured to focus ultrasonic energy emitted by the PMUTs that are disposed on the substrate. Alternatively, or additionally, one or more PMUTs in the array of PMUTs may have a curved surface when in a static position.

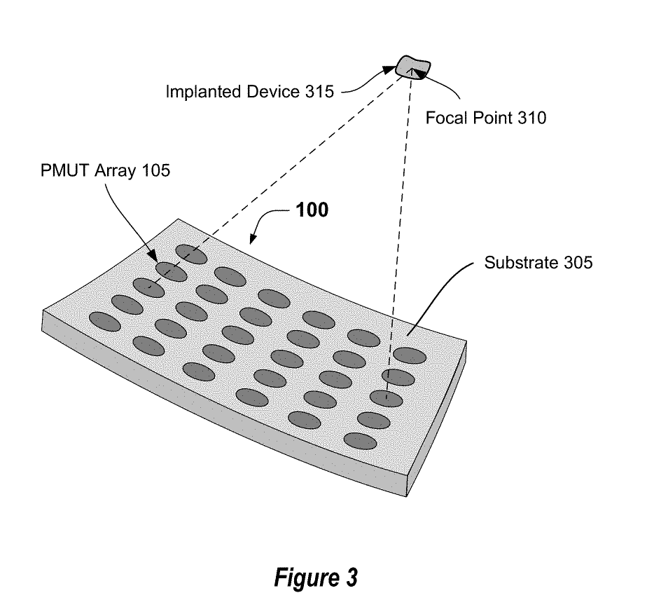

FIG. 3 shows one example of an array of PMUTs residing on a curved substrate. As with other drawings provided herein, the elements of FIG. 3 are not necessarily drawn to scale. In this example, the apparatus 100 includes a substrate 305 having a curvature that is configured for focusing ultrasonic energy emitted by a PMUT array 105 that is disposed on the substrate 305. Here, the curvature of the substrate 305 is configured for focusing ultrasonic energy emitted by the PMUT array 105 at the focal point 310, which corresponds with at least a portion of an implanted device 315 in this example. However, in some instances the focal point 310 may be a first approximation of a target location that corresponds with an implanted device. In some implementations, a coarse-grained adjustment of the position of the focal point 310, including the distance from the substrate 305 to the focal point 310, may be made by adjusting the curvature of the substrate. In alternative implementations, curvature may be attained by disposing layers of the PMUT array 105 onto inwardly curved features (e.g. detents) in the surface of the substrate 305 or by forming a thin and/or flexible substrate 305 and mounting the substrate 305 on a relatively more rigid curved base.

The PMUTs in the PMUT array 105 may have various configurations, depending on the particular implementation. In some examples, one or more PMUTs in the array of PMUTs 105 may have a curved surface when in a static position. Some examples are described elsewhere herein. The PMUTs may or may not include a deformable structural layer, separate from a piezoelectric layer of the PMUT, depending on the particular implementation. This type of deformable structural layer also may be referred to herein as a “mechanical layer.”

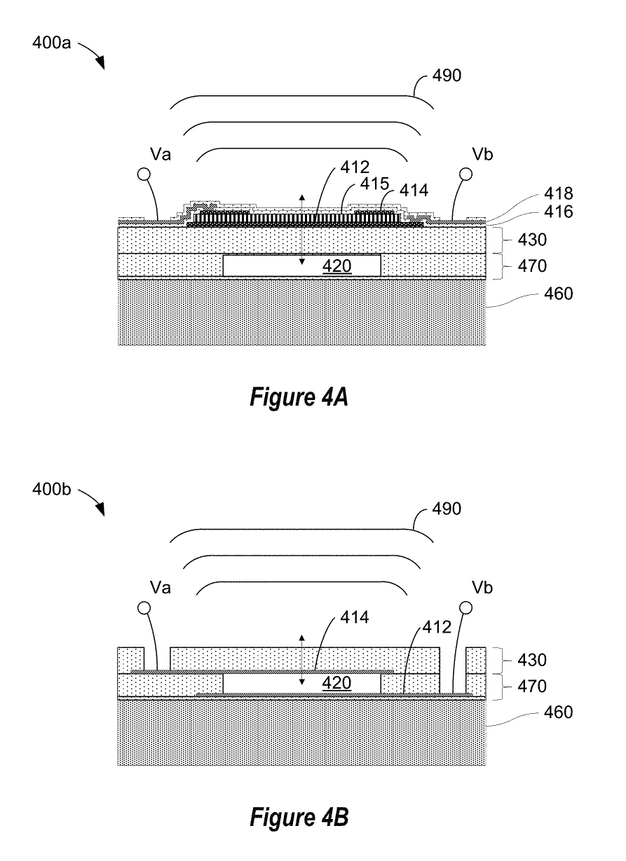

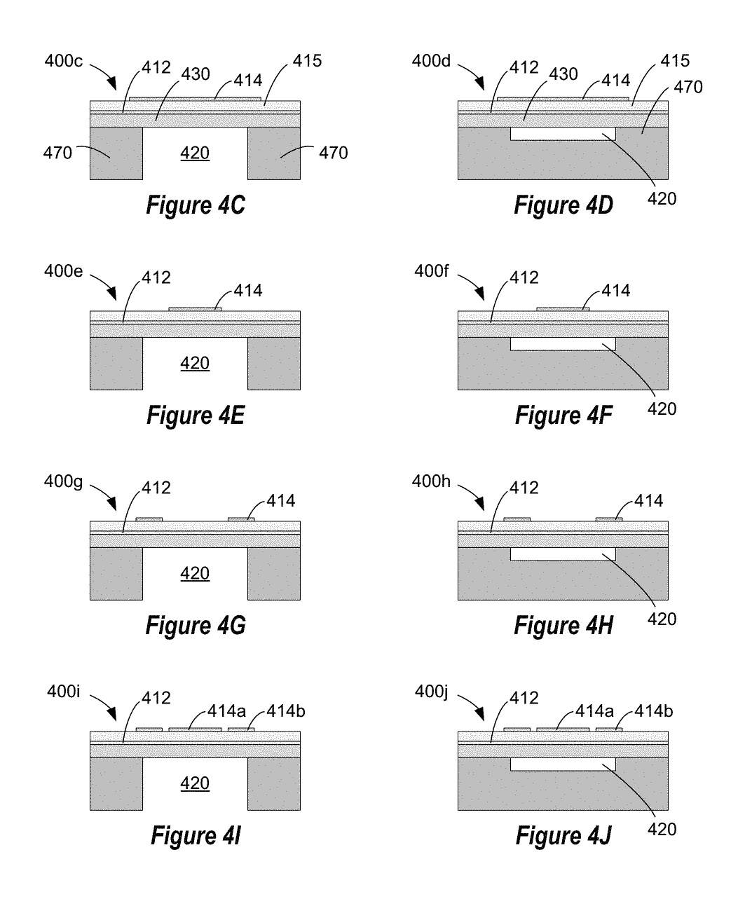

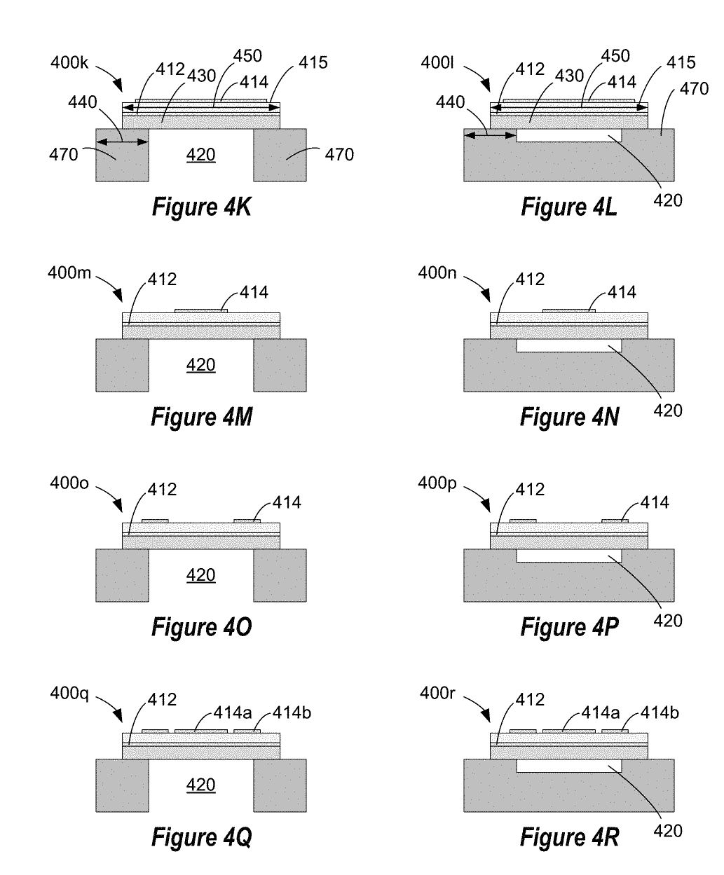

FIG. 4A shows an example of a PMUT element that includes a deformable structural layer that is separate from a piezoelectric layer. The PMUT element 400a may have one or more layers of piezoelectric material such as aluminum nitride (AlN) or lead zirconium titanate (PZT) in a piezoelectric layer that may be used to actuate the PMUT element to generate ultrasonic waves or to detect received ultrasonic waves. The piezoelectric layer stack may include a lower electrode layer 412, a piezoelectric layer 415 and an upper electrode layer 414, with the piezoelectric layer 415 sandwiched between at least a portion of the lower electrode layer 412 and the upper electrode layer 414. The terms “upper” and “lower,” as well as the terms “over,” “under,” “overlying,” “underlying” may be used herein for ease of describing the figures, and indicate relative positions corresponding to the orientation of the figure on a properly oriented page. However, these terms are not limiting and may not reflect the actual orientation or relative positions of elements in a device as implemented. One or more dielectric layers 416 may provide electrical isolation for a metal interconnect layer 418, while allowing connections to lower and upper electrode layers 412 and 414, respectively.

The piezoelectric layer stack may be disposed on, below or above a mechanical layer 430, which is an example of a “deformable structural layer” as used herein. An anchor structure 470 may support the PMUT membrane or diaphragm that is suspended over a cavity 420 and a substrate 460. The substrate 460 may have TFT or CMOS circuitry for driving and sensing the PMUT 400a. The piezoelectric layer stack and mechanical layer 430 may flex, bend or vibrate in response to drive voltages Va and Vb applied across the electrode layers 414 and 412, respectively. Vibrations of the PMUT element 400a may generate ultrasonic waves 490 at a frequency determined by the excitation frequency of the drive voltages. Ultrasonic waves striking the PMUT diaphragm may result in generation of sense voltages Va and Vb with flexing of the diaphragm. The underlying cavity 420 allows for deflections of the PMUT element 400a without contacting the underlying substrate 460. According to some examples, the operating frequencies of the PMUT elements 400a may be tailored for high-frequency operation, low-frequency operation, medium-frequency operation, or a combination of frequencies.

FIG. 4B shows an example of a CMUT element that includes a deformable structural layer without a piezoelectric layer. The CMUT element 400b may have a mechanical layer 430 supported above a cavity 420 and a substrate 460 by an anchor structure 470. Lower electrode 412 on the substrate below the cavity and upper electrode 414 above the cavity 420 may be driven with an excitation voltage applied to terminals Va and Vb to generate ultrasonic waves 490. A potential difference between electrodes 412 and 414 causes an electrostatic force to be generated that attracts the flexible diaphragm of CMUT element 400b downwards towards the substrate. As electrostatic forces are attractive in this configuration whether Va is larger than Vb or Vb is larger than Va, one of the electrodes may need to be biased at a relatively high DC voltage to allow small applied AC voltages to drive the diaphragm up and down. Biasing may also be required for sensing deflections of the CMUT diaphragm above the cavity 420.

PMUT element 400a, while somewhat more complex to fabricate than CMUT element 400b, generally requires smaller operating voltages than the CMUT element 400b to generate similar acoustic power. The PMUT element 400a does not suffer from consequential pull-in voltages for electrostatic devices such as CMUT element 400b, allowing a fuller range of travel. Furthermore, CMUT elements 400b may require significantly higher bias voltages to allow detection of incoming ultrasonic waves.

Although there are some differences between PMUT and CMUT elements, the phrase “PMUT array” may be used herein to refer to an array that includes PMUT elements, CMUT elements, or both PMUT and CMUT elements. In some implementations, the array of PMUTs 105 shown in FIG. 1 may include such a PMUT array. In some implementations, the control system 110 may be capable of addressing at least a portion of the PMUT array for wavefront beam forming, beam steering, receive-side beam forming, and/or selective readout of returned signals. In some implementations, the control system 110 may control at least a portion of the PMUT array to produce wavefronts of a particular shape, such as planar, circular (spherical) or cylindrical wave fronts.

According to some implementations, the control system 110 may be capable of controlling the magnitude and/or phase of at least a portion of the PMUT array to produce constructive or destructive interference in desired locations. For example, the control system 110 may control the magnitude and/or phase of at least a portion of the PMUT array to produce constructive interference towards a target location.

The generation and emission of planar ultrasonic waves (e.g., plane waves) may be achieved by exciting and actuating a large number of PMUT elements in the PMUT array in a simultaneous manner, which may generate an ultrasonic wave with a substantially planar wavefront. Actuation of single PMUT elements in the PMUT array may generate substantially spherical waves in a forward direction, with the PMUT element serving as the source of the spherical waves. Alternatively, the spherical waves may be generated by selecting and exciting an individual PMUT element (e.g., a center element), determining a first ring of PMUT elements around the center PMUT element and actuating the first ring in a delayed manner, determining a second ring of PMUT elements around the first ring and actuating the second ring in a further delayed manner, and so forth as needed. The timing of the excitations may be selected to form a substantially spherical wavefront. Similarly, a cylindrical wave may be generated by selecting and exciting a group of PMUT elements in a row, with the row of PMUT elements serving as the source of the cylindrical waves. Alternatively, the cylindrical waves may be generated by selecting and exciting a row of PMUT elements (the center row), determining and exciting adjacent rows of PMUT elements equidistant from the center row with a controlled time delay, and so forth. The timing of the excitations may be selected to form a substantially cylindrical wavefront.

While exciting an array of PMUT elements simultaneously may produce an ultrasonic plane wave traveling perpendicular to the PMUT array, phase control of PMUT excitation may allow redirection of the plane wave in various directions, depending on the amount of phase delay. For example, if a phase delay of 10 degrees is applied to adjacent rows of PMUT elements that are positioned a distance of one-tenth of a wavelength apart, then the wavefront will transmit a plane wave at an angle of about 15.5 degrees from the normal. Scanning a plane wave at different angles while detecting echoes (reflected portions) from an object positioned in front of the PMUT array may allow detection of the approximate shape, distance and position of the object. Consecutive determinations of object distance and position may allow determination of air gestures.

Other forms of transmit-side beam forming may be utilized. For example, a set of PMUT elements in the PMUT array may be fired in a manner to focus the wavefront of an ultrasonic wave at a particular location in front of the array. For example, the focused wavefront may be cylindrical or spherical by adjusting the timing (e.g., phase) of selected PMUT elements so that the generated wave from each selected PMUT element arrives at a predetermined location in the region in front of the PMUT array at a predetermined time. Focused wavefronts may generate appreciably higher acoustic pressure at a point of interest, and the reflected signal from an object at the point of interest may be detected by operating the PMUT array in a receive mode. The wavefronts emitted from various PMUT elements may interfere constructively in the focal region. The wavefronts from various PMUT elements may interfere destructively in regions near the focal region, providing further isolation of the focused beam energy (amplitude) and increasing the signal-to-noise ratio of the return signal. Similarly, control of the phase at which detection occurs for various PMUT elements in the PMUT array allows receive-side beam forming, in which the return signals may be correlated with distance from a region in space and combined accordingly to generate an image of an object in the detection region. Controlling the frequency, amplitude and phase of the transmitted waves from PMUT elements in the PMUT array may also allow beam shaping and beam forming. In some implementations, not all of the PMUT elements in the PMUT array need be read out for each mode of operation or for each frame. To save processing time and reduce drain on battery life, return signals detected by a select group of PMUT elements may be read out during acquisition. The control system 110 may be configured to address a portion of the PMUT array for wavefront beam forming, beam steering, receive-side beam forming, or selective readout of returned signals.

FIGS. 4C-4R show alternative examples of PMUTs that include a deformable structural layer that is separate from a piezoelectric layer. These implementations are merely examples and alternative implementations are within the scope of the present disclosure. For example, alternative versions of the PMUTs 400c-400r may include a curved piezoelectric layer 415 and/or a curved mechanical layer 430. As with other figures provided herein, the PMUTs 400c-400r are not necessarily drawn to scale. For example, the apparent relative thicknesses of the illustrated layers (such as the relative thicknesses of the lower electrode 412 and the mechanical layer 430) do not necessarily represent the relative thicknesses of the layers in actual PMUTs.

In these examples, each of the PMUTs 400c-400r includes a cavity 420 between anchor structures 470. The PMUTs 400c, 400e, 400g, 400i, 400k, 400m, 400o and 400q include cavity 420 that forms a backside acoustic port, whereas the PMUTs 400d, 400f, 400h, 400j, 400l, 400n, 400p, and 400r include an embedded sealed cavity 420. In some implementations, the backside acoustic port may be used for transmitting and/or receiving ultrasonic waves via transverse displacements of the PMUT diaphragm. In some implementations, the backside acoustic port may aid in forming an acoustic cavity to tailor the acoustic response of the PMUT. In some implementations, the backside acoustic port may be enclosed on some or all sides to tailor the acoustic response of the PMUT. The examples shown in FIGS. 4C-4R include a piezoelectric layer 415 that spans the region of the cavity 420. The examples shown in FIGS. 4C-4R also include a deformable structural layer (the mechanical layer 430) that spans the cavity region of the cavity 420.

In this example, each of the PMUTs 400c-400r includes a piezoelectric layer 415, a first electrode on a first side of the piezoelectric layer 415 and a second electrode on a second side of the piezoelectric layer 415. The “first electrode” and the “second electrode” may be the lower electrode 412 and the upper electrode 414, respectively, or vice versa. The piezoelectric layer 415, the first electrode and the second electrode reside on a support structure, which corresponds with the anchor structures 470 in this example.

In the examples of FIGS. 4C and 4D, the upper electrode 414 is a center electrode. Here, the PMUTs 400c and 400d include a lower electrode 412 and an upper electrode 414 that span the entire cavity region of the cavity 420. Such implementations may be referred to as having “full diaphragm electrodes.” Some such implementations may have a relatively lower coupling factor, as compared to the PMUTs 400e-400j. Configurations 400c and 400d with full diaphragm electrodes rely in part on in-plane expansion and contraction of the piezoelectric layer 415 and the structural layer 430 to generate edge moments that can cause transverse (e.g., upward and downward) motions of the PMUT diaphragm and generate ultrasonic waves.

In FIGS. 4E and 4F, the upper electrode 414 is a center electrode that does not span the entire region of the cavity 420. However, in these examples the PMUTs 400e and 400f include a lower electrode 412 that spans the region of the cavity 420. Some such implementations may have a relatively higher coupling factor, as compared to the PMUTs 400c and 400d, but a relatively lower coupling factor than that of PMUTs 400i and 400j. Drive signals applied to the center electrode generate mechanical bending moments within the PMUT diaphragm and cause transverse motions of the diaphragm.

In FIGS. 4G and 4H, the upper electrode 414 is a ring electrode. In these examples, the upper electrode 414 resides partially over the cavity 420 and partially over the anchor structures 470. Accordingly, the upper electrodes 414 do not span the entire region of the cavity 420. However, in these examples the PMUTs 400e and 400f include a lower electrode 412 that spans the region of the cavity 420. Some such implementations may have a relatively higher coupling factor, as compared to the PMUTs 400c and 400d, but a relatively lower coupling factor than that of the PMUTs 400i and 400j. Drive signals applied to the ring electrode generate mechanical bending moments within the PMUT diaphragm and cause transverse motions of the diaphragm in a manner similar to that of PMUTs 400c and 400d, yet in an opposite direction.

In FIGS. 4I and 4J, the upper electrode 414a is a center electrode that does not span the entire region of the cavity 420 and the upper electrode 414b is a ring electrode that resides partially over the cavity 420 and partially over the anchor structures 470. Accordingly, the upper electrodes 414a and 414b do not span the entire region of the cavity 420. However, in these examples the PMUTs 400e and 400f include a lower electrode 412 that spans the region of the cavity 420. Some such implementations may have a relatively higher coupling factor, as compared to the PMUTs 400c-400h. Drive signals applied to the center electrode and drive signals of an opposite polarity applied simultaneously to the ring electrode augment each other to increase the magnitude of the transverse motions of the PMUT diaphragm and therefore increase the amplitude of the generated ultrasonic waves.

In the examples of FIGS. 4K and 4L, the upper electrode 414 is a center electrode. Here, the PMUTs 400k and 400l include a lower electrode 412 and an upper electrode 414 that span the entire cavity region of the cavity 420. Accordingly, the PMUTs 400k and 400l are similar to the PMUTs 400c and 400d. Likewise, the upper and lower electrode arrangements of the PMUTs 400m and 400n are similar to those of the PMUTs 400e and 400f, the upper and lower electrode arrangements of the PMUTs 400o and 400p are similar to those of the PMUTs 400g and 400h, and the upper and lower electrode arrangements of the PMUTs 400q and 400r are similar to those of the PMUTs 400i and 400j.

However, one significant difference between the PMUTs 400k-400r and the PMUTs 400c-400j is that in the PMUTs 400k-400r, at least a portion of a support structure area extends beyond an area of the piezoelectric layer 415. Referring to FIGS. 4K and 4L, for example, the arrows 440 indicate the areas of the support structures 470 and the arrows 450 indicate the areas of the piezoelectric layers 415. One may observe that in each of the PMUTs 400k and 400l, a portion of a support structure area extends beyond an area of the piezoelectric layer 415. This is also true for the PMUTs 400m-400r. Put another way, in the PMUTs 400k-400r, the area of the piezoelectric layers 415 extends over only a portion of the areas of the support structures 470.

This configuration produces a relatively higher edge moment for the PMUTs 400k-400r, as compared to the PMUTs 400c-400j. Having a higher edge moment is potentially advantageous because higher edge moments can generate larger deflections of the PMUT diaphragm and therefore generate ultrasonic waves with a higher amplitude.

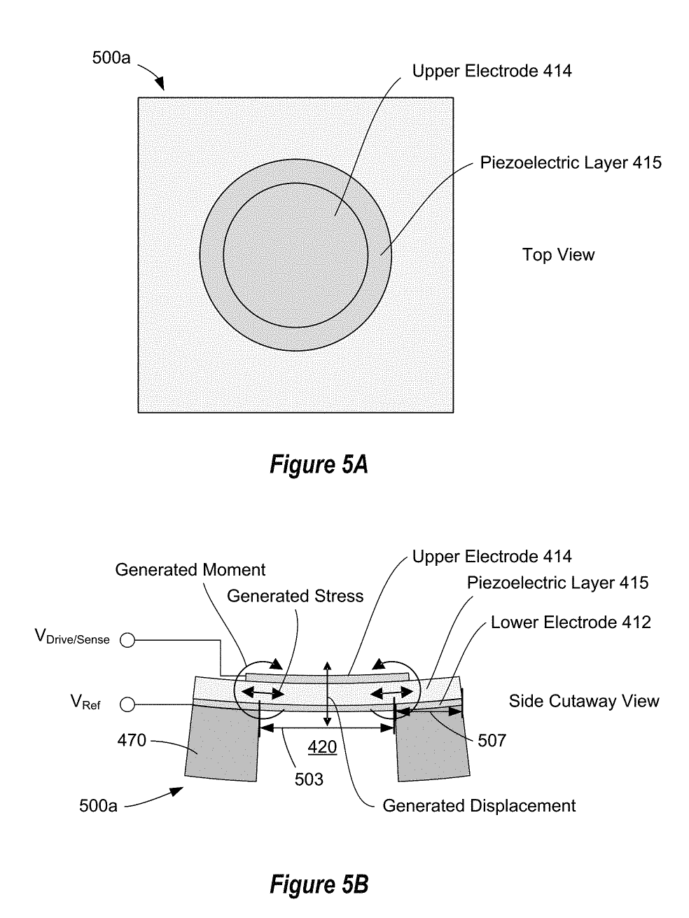

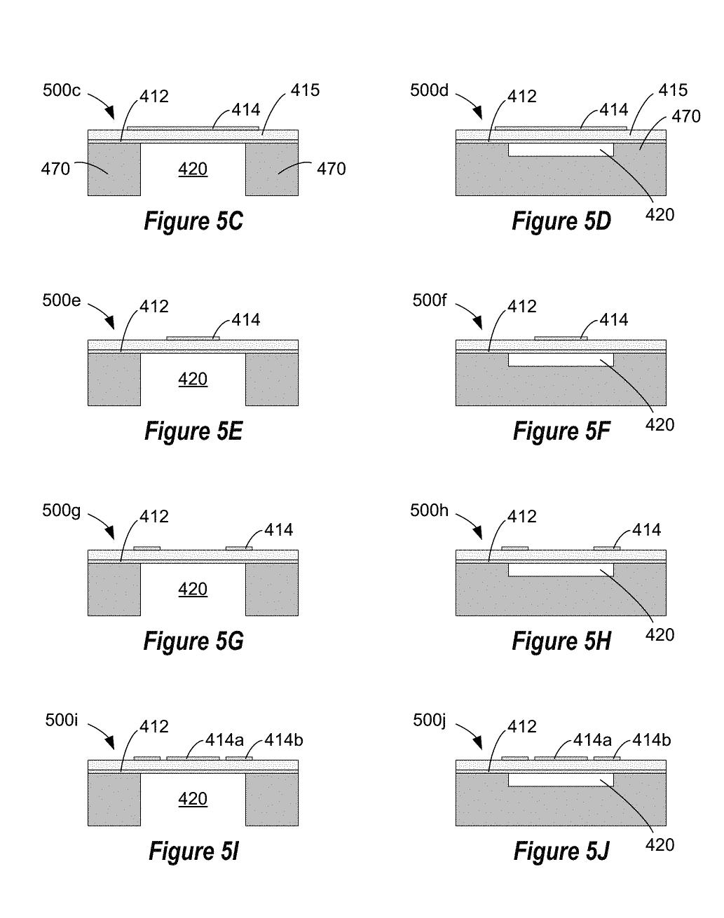

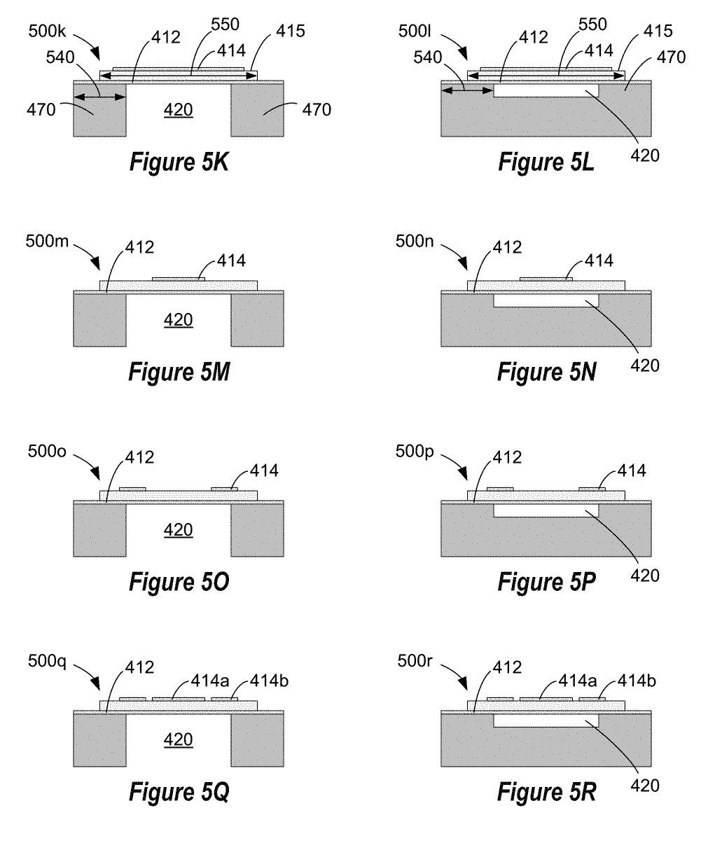

FIGS. 5A and 5B show a top view and a cross-sectional view, respectively, of one example of a PMUT having a curved surface when in a rest position. In this example, the PMUT 500a includes a piezoelectric layer 415, a first electrode on a first side of the piezoelectric layer 415 and a second electrode on a second side of the piezoelectric layer 415. The “first electrode” and the “second electrode” may be the lower electrode 412 and the upper electrode 414, respectively, or vice versa. In this example, both the first electrode and the second electrode span a region of the cavity 420. The piezoelectric layer 415, the first electrode and the second electrode reside on a support structure, which corresponds with the anchor structures 470 in this example. In this example, as shown in FIG. 5A, the upper electrode 414 is a center electrode.

Unlike the examples shown in FIGS. 4A-4R, the PMUT 500a includes no deformable structural layer that is separate from the piezoelectric layer 415. For example, there is no mechanical layer 430 proximate a first side or a second side of the piezoelectric layer 415. Instead, acoustic waves (such as ultrasonic waves) can be generated by displacement of the piezoelectric layer 415 itself.

In this example, the piezoelectric layer 415 has an initial non-zero curvature when the PMUT is in a “rest” position, with no drive voltage applied. In the example shown in FIG. 5B, a first portion 503 of the piezoelectric layer 415 spans a region of the cavity 420 and a second portion 507 of the piezoelectric layer 415 is mechanically coupled to a support structure (here, the anchor structure 470) adjacent the region of the cavity 420. In this example, the second portion 507 of the piezoelectric layer 415 and the support structure combine to produce a mechanical moment on the first portion 503 of the piezoelectric layer 415 when a transmitter excitation signal is applied to the first electrode or the second electrode. Accordingly, an applied drive voltage results in bending moments and transverse deflections or displacements via piezoelectric layer expansion and contraction. In this example, the transverse displacement is along a radius of curvature of the piezoelectric layer 415, whereas the generated stress is along an arc having a curvature of the piezoelectric layer 415.

FIGS. 5C-5R show alternative examples of PMUTs that do not include a deformable structural layer that is separate from a piezoelectric layer. These implementations are merely examples and alternative implementations are within the scope of the present disclosure. For example, alternative versions of the PMUTs 500c-500r may include a piezoelectric layer 415 with an initial non-zero curvature (not shown) when the PMUT is in a “rest” position, with no drive voltage applied. As with other figures provided herein, the PMUTs 500c-500r are not necessarily drawn to scale.

Here, the PMUTs 500c-500r all include a piezoelectric layer 415, a first electrode on a first side of the piezoelectric layer 415 and a second electrode on a second side of the piezoelectric layer. The “first electrode” and the “second electrode” may be the lower electrode 412 and the upper electrode 414, respectively, or vice versa. The piezoelectric layer 415, the first electrode and the second electrode reside on a support structure, which corresponds with the anchor structures 470 in this example.

Like the implementation shown in FIG. 5B, the PMUTs 500c-500r include no deformable structural layer that is separate from the piezoelectric layer 415. For example, there is no mechanical layer 430 proximate a first side or a second side of the piezoelectric layer 415. Instead, acoustic waves (such as ultrasonic waves) can be generated by displacement of the piezoelectric layer 415 itself.

In these examples, each of the PMUTs 500c-500r includes a cavity 420 between anchor structures 470. The PMUTs 500c, 500e, 500g, 500i, 500k, 500m, 500o and 500q include cavity 420 that forms a backside acoustic port, whereas the PMUTs 500d, 500f, 500h, 500j, 500l, 500n, 500p, and 500r include an embedded sealed cavity 420.

In the examples of FIGS. 5C and 5D, the upper electrode 414 is a center electrode configured as a full diaphragm electrode. Here, the PMUTs 500c and 500d include a lower electrode 412 and an upper electrode 414 that span the entire cavity region of the cavity 420. Some such implementations may have a relatively higher coupling factor, as compared to the PMUTs 500e-500j. This is due, at least in part, to the relatively larger portion of the piezoelectric layer 415 that is in contact with the lower electrode 412 and the upper electrode 414, which allows a significant portion of the piezoelectric layer 415 to be activated when transmitter excitation signals are applied to upper electrode 414 and lower electrode 412 and therefore can generate a more intense ultrasonic wave.

In FIGS. 5E and 5F, the upper electrode 414 is a center electrode that does not span the region of the cavity 420. However, in these examples the PMUTs 500e and 500f include a lower electrode 412 that spans the region of the cavity 420. Some such implementations may have a relatively lower coupling factor, as compared to the PMUTs 500c, 500d, 500i and 500j. Transmitter excitation signals applied to the center electrode generate in-plane stresses and resulting mechanical bending moments within the PMUT diaphragm, thereby generating transverse motions of the diaphragm.

In FIGS. 5G and 5H, the upper electrode 414 is a ring electrode. In these examples, the upper electrode 414 resides partially over the cavity 420 and partially over the anchor structures 470. Accordingly, the upper electrodes 414 do not span the entire region of the cavity 420. However, in these examples the PMUTs 500g and 500h include a lower electrode 412 that spans the entire region of the cavity 420. Transmitter excitation signals applied to the ring electrode generate in-plane stresses and resulting mechanical bending moments within the PMUT diaphragm, thereby generating transverse motions of the diaphragm. Some such implementations may have a relatively lower coupling factor, as compared to the PMUTs 500c, 500d, 500i and 500j.

In FIGS. 5I and 5J, the upper electrode 414a is a center electrode that does not span the entire region of the cavity 420 and the upper electrode 414b is a ring electrode that resides partially over the cavity 420 and partially over the anchor structures 470. Accordingly, the upper electrodes 414a and 414b do not span the entire region of the cavity 420. However, in these examples the PMUTs 500e and 500f include a lower electrode 412 that spans the region of the cavity 420. Some such implementations may have a relatively higher coupling factor, as compared to the PMUTs 500e-500h, but a relatively lower coupling factor, as compared to the PMUTs 500c and 500d.

In the examples of FIGS. 5K and 5L, the upper electrode 414 is a center electrode. Here, the PMUTs 500k and 500l include a lower electrode 412 and an upper electrode 414 that span the entire cavity region of the cavity 420. Such implementations may have a relatively higher coupling factor, as compared to the PMUTs 500m-500r. Accordingly, the PMUTs 500k and 500l are similar to the PMUTs 500c and 500d. Likewise, the upper and lower electrode arrangements of the PMUTs 500m and 500n are similar to those of the PMUTs 500e and 500f, the upper and lower electrode arrangements of the PMUTs 500o and 500p are similar to those of the PMUTs 500g and 500h, and the upper and lower electrode arrangements of the PMUTs 500q and 500r are similar to those of the PMUTs 500i and 500j.

However, one significant difference between the PMUTs 500k-500r and the PMUTs 500c-500j is that in the PMUTs 500k-500r, at least a portion of a support structure area extends beyond an area of the piezoelectric layer 415. Referring to FIGS. 5K and 5L, for example, the arrows 540 indicate the area of the support structures 470 and the arrows 550 indicate the area of the piezoelectric layers 415. One may observe that in the PMUTs 500k and 500l, a portion of a support structure area extends beyond an area of the piezoelectric layer 415. This is also true for the PMUTs 500m-500r. Put another way, in the PMUTs 500k-500r, the area of the piezoelectric layers 415 extends over only a portion of the areas of the support structures 470.

This configuration produces a relatively higher edge moment for the PMUTs 500k-500r, as compared to the PMUTs 500c-500j. Having a higher edge moment is potentially advantageous because higher edge moments can generate larger deflections of the PMUT diaphragm and therefore generate ultrasonic waves with a higher amplitude.

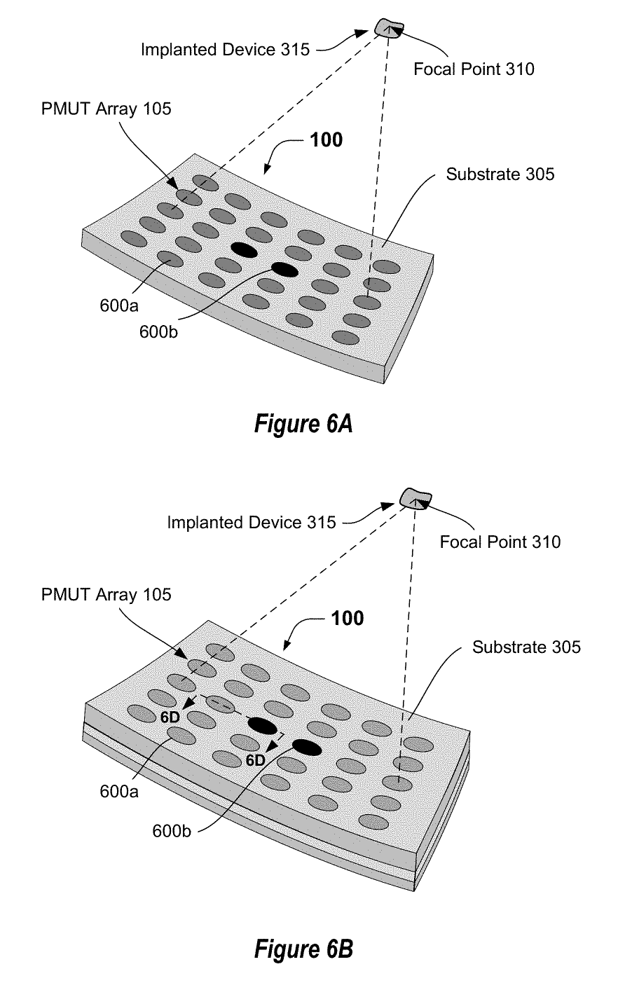

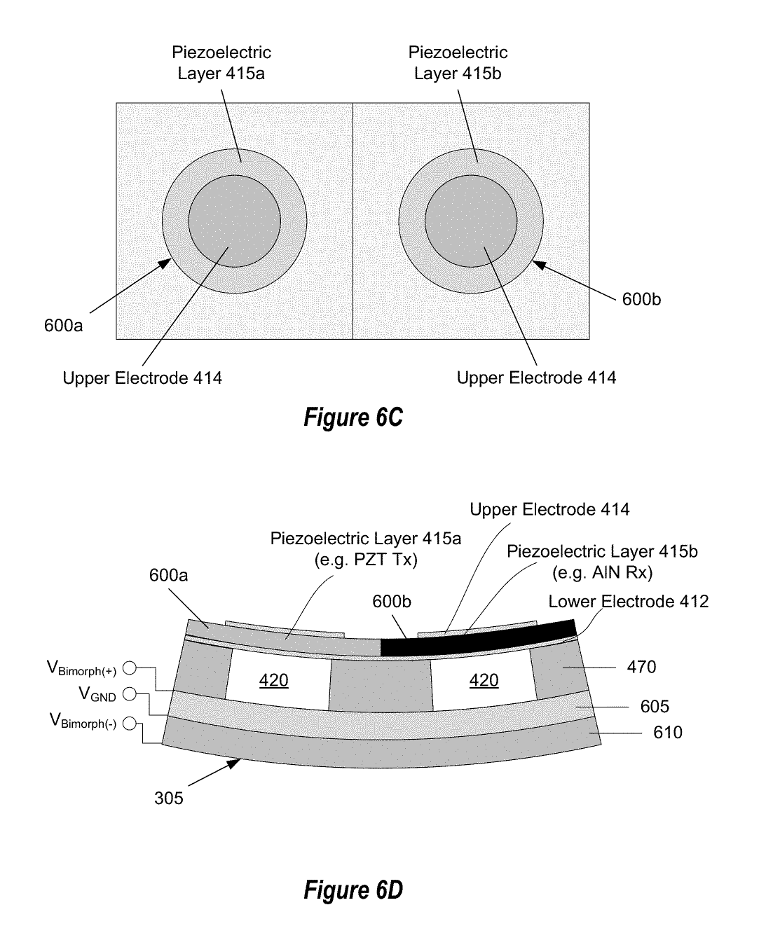

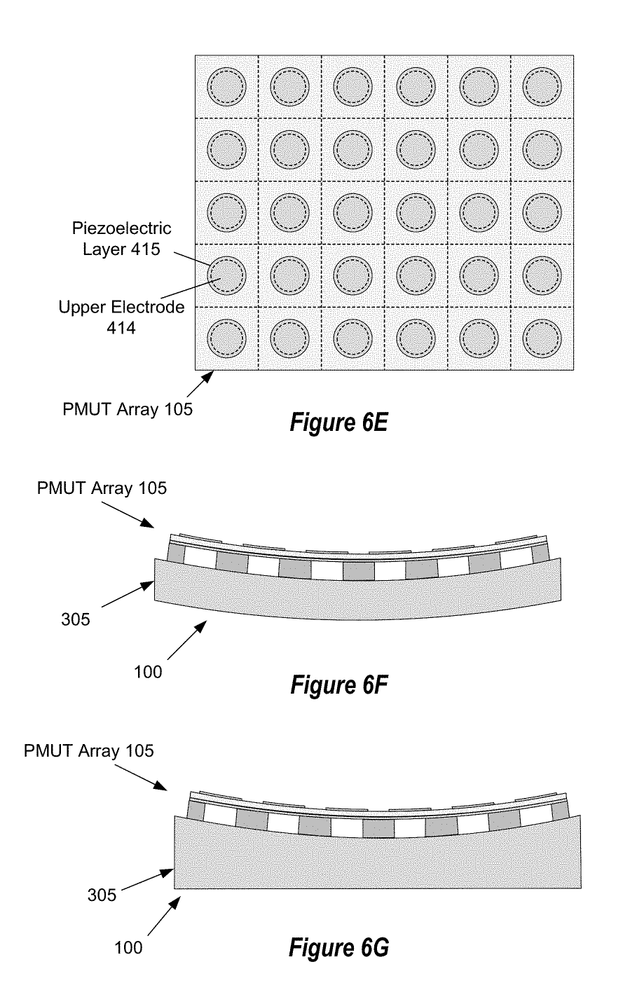

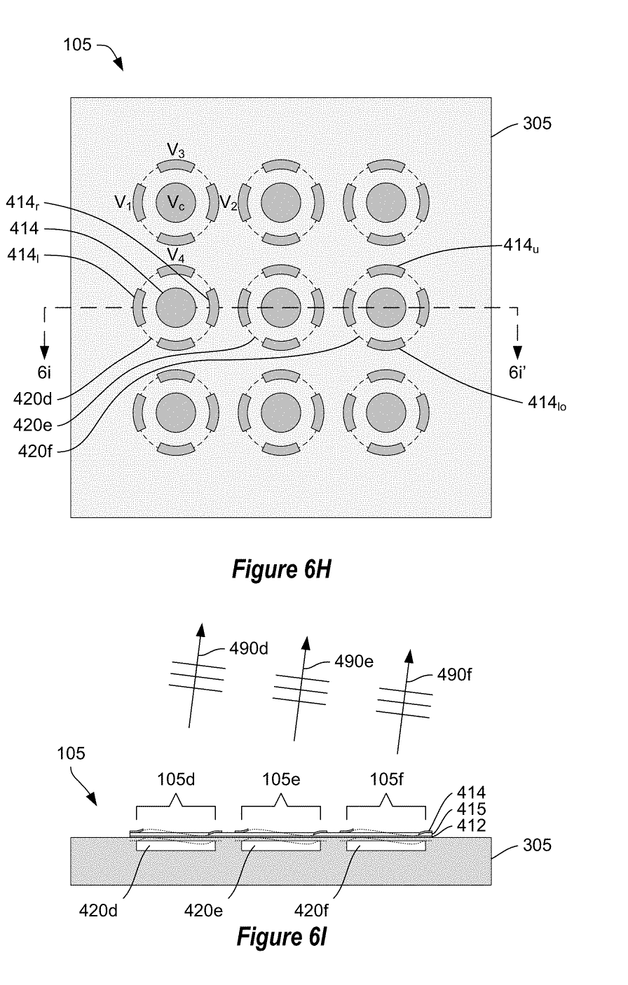

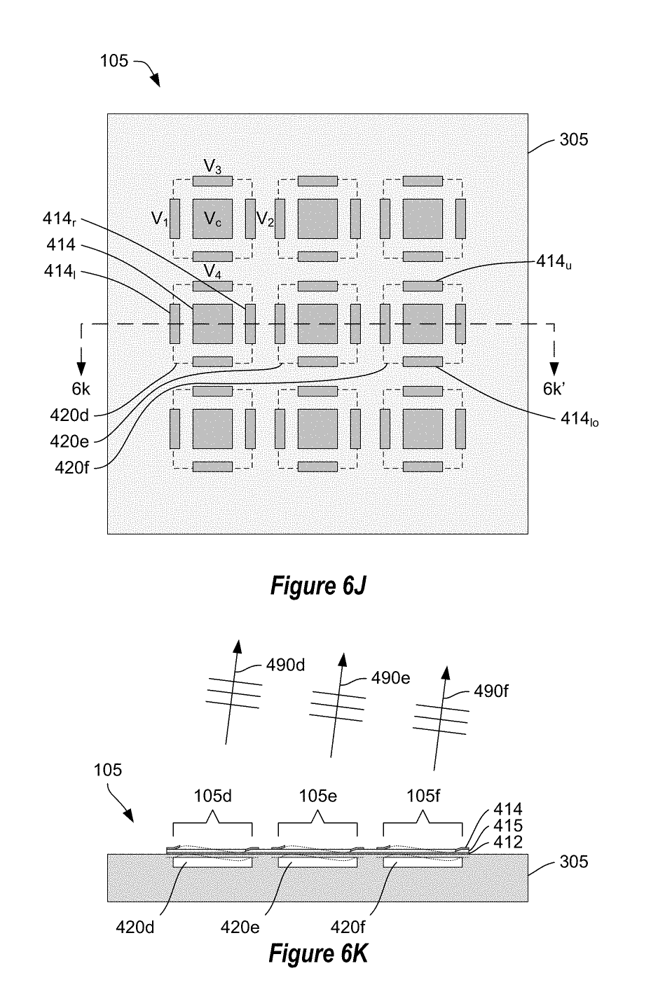

FIG. 6A shows an example of a PMUT array including PMUTs that are configured for detecting received ultrasonic energy. In this example, one or more PMUTs 600a are configured for transmitting ultrasonic waves and one or more PMUTs 600b are configured for receiving ultrasonic waves, with PMUTs 600b configured for receiving ultrasonic waves positioned near the center of the PMUT array 105. The particular arrangements of PMUTs shown in FIG. 6A (and each of the other disclosed drawings) are merely examples. In alternative implementations, the PMUTs that are configured for detecting received ultrasonic energy may be more numerous and/or positioned in different areas of the PMUT array 105.

According to some examples, determining a target location may be based, at least in part, on ultrasonic waves that are reflected from or transmitted from the target location and received by the PMUTs 600b. In some such examples, a control system of the apparatus 100 may be configured to control the PMUTs 600a to scan a region inside a human body with transmitted ultrasonic waves. For example, controlling the PMUTs 600a to scan the region inside the human body with transmitted ultrasonic waves may involve changing a curvature of the substrate 305, controlling the PMUTs 600a for beam steering and/or for changing an orientation of one or more PMUT diaphragms. In some such implementations, determining the target location may be based, at least in part, on ultrasonic waves that are reflected from the target location and received by the PMUTs 600b.

In this example, the apparatus 100 includes a substrate 305 having a curvature that is configured for focusing ultrasonic energy emitted by a PMUT array 105 that is disposed on the substrate 305. Here, the curvature of the substrate 305 is configured for focusing ultrasonic energy emitted by the PMUTs at the focal point 310, which corresponds with at least a portion of the implanted device 315 in this example.

According to some such examples, the PMUTs that are configured for transmitting ultrasonic energy (in this example, the PMUTs 600a) may include a different type of piezoelectric material than the PMUTs that are configured for detecting received ultrasonic energy (in this example, the PMUTs 600b). For example, the PMUTs that are configured for transmitting ultrasonic energy may be formed of a piezoelectric material (such as lead zirconate titanate (PZT)) having a higher transverse piezoelectric coefficient (d31) and/or having a higher dielectric constant relative to that of the piezoelectric material of the PMUTs that are configured for detecting received ultrasonic energy. The latter type of piezoelectric material may, for example, be aluminum nitride (AlN). In some examples, the PMUTs that are configured for transmitting ultrasonic energy may be formed of a piezoelectric material having a smaller thickness relative to the piezoelectric material of the PMUTs configured to detect received ultrasonic waves.

FIG. 6B shows another example of a PMUT array including PMUTs that are configured for detecting received ultrasonic energy. In this example, the PMUTs 600a are configured for transmitting ultrasonic waves and the PMUTs 600b are configured for receiving ultrasonic waves. According to this implementation, a coarse-grained adjustment of the position of the focal point 310, including the distance from at least a portion of the substrate 305 to the focal point 310, may be made by adjusting the curvature of the substrate 305.

FIGS. 6C and 6D show a top view and a cross-sectional view, respectively, of a portion of the apparatus shown in FIG. 6B. The dashed arrow shown in FIG. 6B indicates the approximate location through which the cross-sectional view of FIG. 6D is taken.

In this example, the PMUTs 600a and 600b have a curved surface when in a rest position. In this example, the PMUTs 600a and 600b includes piezoelectric layers 415a and 415b, respectively, a first electrode on a first side of the piezoelectric layers 415a and 415b and a second electrode on a second side of the piezoelectric layers 415a and 415b. The “first electrode” and the “second electrode” may be the lower electrode 412 and the upper electrode 414, respectively, or vice versa. In this example, both the first electrode and the second electrode span a region of the cavity 420. The piezoelectric layers 415a and 415b, the first electrode and the second electrode reside on a support structure, which corresponds with the anchor structures 470 in this example. In this example, the upper electrode 414 is a center electrode.

Like the examples shown in FIGS. 5A-5R, the PMUTs 600a and 600b include no deformable structural layer that is separate from the piezoelectric layers 415a and 415b. In this example, the piezoelectric layers 415a and 415b have an initial non-zero curvature when the PMUT 600a is in a “rest” position, with no drive voltage applied. Here, first portions of the piezoelectric layers 415a and 415b span a region of the cavities 420 and second portions of the piezoelectric layers 415a and 415b are mechanically coupled to a support structure (here, the anchor structure 470) adjacent the region of the cavities 420. In this example, the second portion of the piezoelectric layer 415a and the support structure combine to produce a mechanical moment on the first portion 503 of the piezoelectric layer 415a when a transmitter excitation signal is applied. Accordingly, an applied drive voltage results in bending moments and transverse displacement via piezoelectric layer expansion and contraction. In this example, the transverse displacement is along a radius of curvature of the piezoelectric layer 415a, whereas the generated stress is along an arc having a curvature of the piezoelectric layer 415a.

In the implementation shown in FIG. 6D, the substrate 305 includes piezoelectric layers 605 and 610. Piezoelectric layers 605 and 610 may combine to form a bimorph actuator, which may bend in one direction or the other when suitable actuation voltages are applied. Piezoelectric layers 605 and 610 may include PZT, AlN or other piezoelectric material. By causing a voltage (shown in FIG. 6D as V_{Bimorph+}) to be applied across the layer 605, a control system may cause the curvature of the substrate 305 to change in one sense (either increasing or decreasing). By causing a voltage (shown in FIG. 6D as V_{Bimorph-}) to be applied across the piezoelectric layer 610, a control system may cause the curvature of the substrate 305 to increase further in one direction or the other. Switching the polarities of actuation voltages V_{Bimorph+} and V_{Bimorph-} causes curvature of the substrate 305 in an opposite direction. In this manner, the control system can make a coarse-grained adjustment of the position of the focal point of a PMUT array 105 that includes the PMUTs 600a and 600b. In alternative configurations, a single piezoelectric layer 605 or 610 may be coupled to a metal or non-metal backing layer of non-piezoelectric material such that an actuation voltage applied across the piezoelectric layer induces a curvature in the piezoelectric layer 605 or 60, backing layer and attached substrate 305.

As noted elsewhere herein, determining a target location may be based, at least in part, on ultrasonic waves that are reflected from or transmitted from the target location and received by the PMUTs 600b. In some such examples, a control system may be configured to control the PMUTs 600a to scan a region inside a human body with transmitted ultrasonic waves. According to some implementations, controlling the PMUTs 600a to scan the region inside the human body with transmitted ultrasonic waves may involve causing the curvature of the substrate 305 to change, as described above. Alternatively, or additionally, controlling the PMUTs 600a to scan the region inside the human body with transmitted ultrasonic waves may involve controlling the PMUTs 600a for beam steering and/or for changing an orientation of one or more PMUT diaphragms. In some such implementations, determining the target location may be based, at least in part, on ultrasonic waves that are reflected from the target location and received by the PMUTs 600b.

FIGS. 6E and 6F show a top view and a cross-sectional view, respectively, of an example of an implementation of a PMUT array configured for detecting, receiving and transmitting ultrasonic energy. In the example shown in FIG. 6F, the apparatus 100 includes a relatively thin and/or flexible PMUT array 105 residing on a relatively more rigid, curved substrate 305. Here, both the upper surface and the lower surface of the substrate 305 are curved.

FIG. 6G shows a cross-sectional view of another implementation of a PMUT array configured for detecting, receiving and transmitting ultrasonic energy. Like the example shown in FIG. 6F, the apparatus 100 includes a relatively thin and/or flexible PMUT array 105 residing on a relatively more rigid, curved substrate 305. However in this example, the upper surface is curved and the lower surface is not curved. In alternative implementations, the PMUT array 105 may reside on and may have been formed on a thin and flexible substrate 305 that is later mounted on a thicker, relatively more rigid and curved substrate like the one shown in FIG. 6F or 6G.

FIG. 6H shows a plan view of an implementation of a PMUT array configured for changing an orientation of one or more PMUT diaphragms in the PMUT array 105. The PMUTs in the PMUT array 105 as shown have circular diaphragms on a substrate 305 and are configured in a three-by-three array for clarity, although other array sizes and geometries including linear, square, rectangular and close-packed hexagonal arrays have been contemplated. Each PMUT in the PMUT array 105 may include one or more edge electrodes for controlling the static displacement, tilt and curvature of the associated PMUT diaphragm when proper directional control voltages are applied. Each of the round PMUT diaphragms as shown in FIG. 6H may include an upper edge electrode 414ᵤ, a lower edge electrode 414ₗₒ, a left edge electrode 414ₗ and a right edge electrode 414ᵣ for controlling the orientation of the PMUT diaphragm and a center electrode 414 for generating dynamic displacements of the PMUT diaphragm and transmitting ultrasonic waves. Orientation and directional control voltages V₁, V₂, V₃ and V₄ may be applied to the left edge electrode 414ₗ, right edge electrode 414ᵣ, the upper edge electrode 414ᵤ and the lower edge electrode 414ₗₒ, respectively, to control the orientation of the PMUT diaphragm and the direction of transmitted ultrasonic waves from one or more PMUTs in the PMUT array 105. A transmitter excitation voltage V_{c} may be applied to the center electrode to generate the ultrasonic waves.

FIG. 6I shows a cross-sectional view through line 6i-6i′ of the implementation shown in FIG. 6H, with the PMUT array configured for changing the orientation of one or more PMUT diaphragms and for beam steering. One or more PMUT diaphragms may be controlled by a control system 110 (not shown) to change an orientation of the PMUT diaphragm towards a target location and to focus ultrasonic energy at the target location. For example, PMUT diaphragms 105d, 105e and 105f above embedded cavities 420d, 420e and 420f, respectively, are oriented with orientation control voltages applied to the left and right edge electrodes and may generate ultrasonic waves 490d, 490e and 490f, respectively when transmitter excitation signals are applied to the center electrodes 414. In some implementations, a positive control voltage may be applied to the left edge electrodes 414l of PMUT diaphragms 105d, 105e and 105f and a negative control voltage may be applied to the right edge electrodes 414r of PMUT diaphragms 105d, 105e and 105f to achieve an upwardly bending moment on one edge (e.g., the left side) and a downwardly bending moment on an opposite edge (e.g., the right side) that results in a rightward-tilted orientation of the PMUT diaphragms 105d, 105e and 105f as illustrated in FIG. 6I, which may be oriented towards a target location. Transmitter excitation signals applied to center electrodes 414 of PMUT diaphragms 105d, 105e and 105f may be appropriately phased in a beam steering process to focus and constructively reinforce ultrasonic energy at the target location.

FIG. 6J shows a plan view of another implementation of a PMUT array configured for changing an orientation of one or more PMUT diaphragms in the PMUT array. In this example, the PMUTs of the PMUT array 105 include square cavities 420 and rectangular edge electrodes for controlling the orientation of the PMUT diaphragm when proper control voltages are applied in a manner similar to the circular PMUTs in the PMUT array 105 shown and described with respect to FIG. 6H.

FIG. 6K shows a cross-sectional view through line 6k-6k′ of the implementation shown in FIG. 6J, with the PMUT array configured for changing the orientation of one or more PMUT diaphragms and for beam steering to focus ultrasonic energy at a target location in a manner similar to the circular PMUTs in the PMUT array 105 shown and described with respect to FIG. 6I.

In some implementations, a positive (or negative) control voltage of the same polarity may be applied to each of the edge electrodes of one or more PMUT diaphragms to generate a static curvature of the diaphragm in an upwards or downwards direction, which may aid in focusing ultrasonic energy towards a target location.

As noted elsewhere herein, according to some implementations a control system of the apparatus 100 may be configured for determining a target location within a human body and for controlling the array of PMUTs 105 to focus ultrasonic energy at the target location. In some such examples, determining the target location may be based, at least in part, on received signals, which may be received ultrasonic signals.

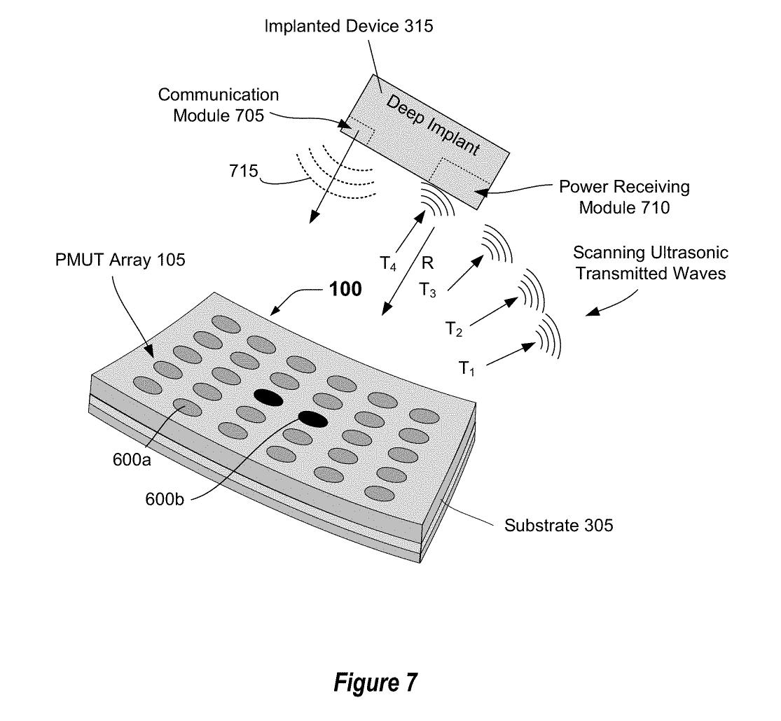

FIG. 7 shows an example of apparatus configured for determining a target location within a human body. In the example shown in FIG. 7, a control system of the apparatus 100 has caused at least some PMUTs 600a in the array of PMUTs 105 to broadly scan a region with transmitted ultrasonic waves in which a deeply-implanted device may be positioned. In some such examples, the array of PMUTs 105 may reside on a wearable device or in a shallow implanted device. The scanning process may involve one or more of changing a curvature of a substrate on which the array of PMUTs 105 resides, performing a beam steering process, or changing an orientation of one or more PMUT diaphragms.

The implanted device 315 may or may not include the optional communication module 705, depending on the particular implementation. In some implementations wherein the implanted device 315 includes a communication module 705, the communication module 705 may need at least a small amount of power to be activated. This power may, in some examples, be provided by the scan of transmitted ultrasonic waves from the apparatus 100.

In a first example, the implanted device 315 includes a communication module 705. As shown in FIG. 7, a control system of the apparatus 100 has caused at least some PMUTs 600a to transmit ultrasonic waves in a direction T₁ at a first time during a scanning process. The transmissions in the direction T₁ did not result in any response from the communication module 705, so the scanning process continued: the control system of the apparatus 100 caused at least some PMUTs 600a to transmit ultrasonic waves in a direction T₂ at a second time. The transmissions in the direction T₂ did not result in any response from the communication module 705, so the scanning process continued: the control system of the apparatus 100 caused at least some PMUTs 600a to transmit ultrasonic waves in a direction T₃ at a third time.

Once again, the transmissions in the direction T₃ did not result in any response from the communication module 705, so the scanning process continued: the control system of the apparatus 100 caused at least some PMUTs 600a to transmit ultrasonic waves in a direction T₄ at a fourth time. These ultrasonic waves were received by, and transmitted a small amount of power to, the power receiving module 710. In this example, the power receiving module 710 received enough power to activate the communication module 705.

Therefore, the communication module 705 has transmitted the signals 715, which may be detected by the apparatus 100. In some examples, the communication module 705 may be capable of transmitting acoustic waves, such as ultrasonic waves, that may be detected by at least some PMUTs (such as the PMUTs 600b) of the PMUT array 105. In some implementations, the power receiving module 710 and the communication module 705 may include PMUT arrays for communication and for power transfer.

However, in alternative implementations the communication module 705 may be capable of transmitting other types of signals, such as electromagnetic signals. The apparatus 100 may include a receiver capable of detecting such signals.

In some implementations, the communication module 705 may send power transfer information to the apparatus 100. The power transfer information may include information for facilitating and/or optimizing a power transfer process. For example, the power transfer information may indicate an ultrasonic wave intensity level, an ultrasonic frequency or frequency range for power transmission, an implanted device type, a power receiving module type, an estimated depth and/or position of the implanted device within a human body, etc.

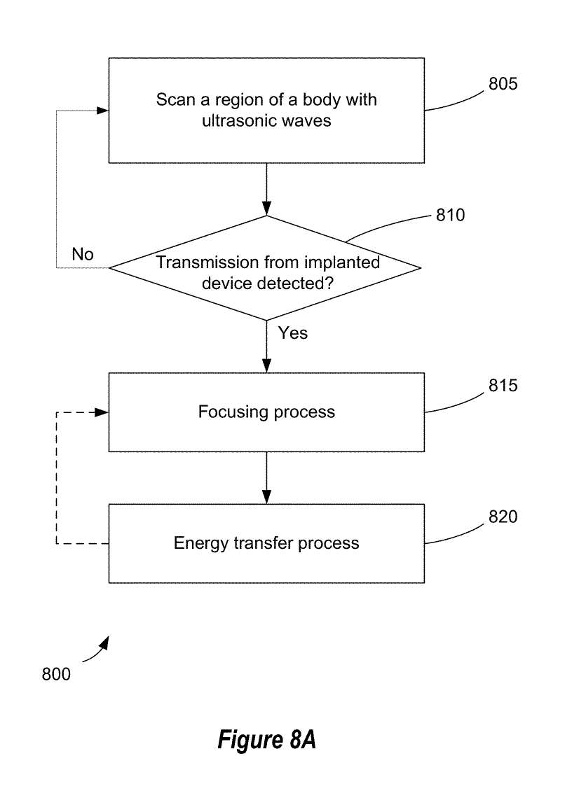

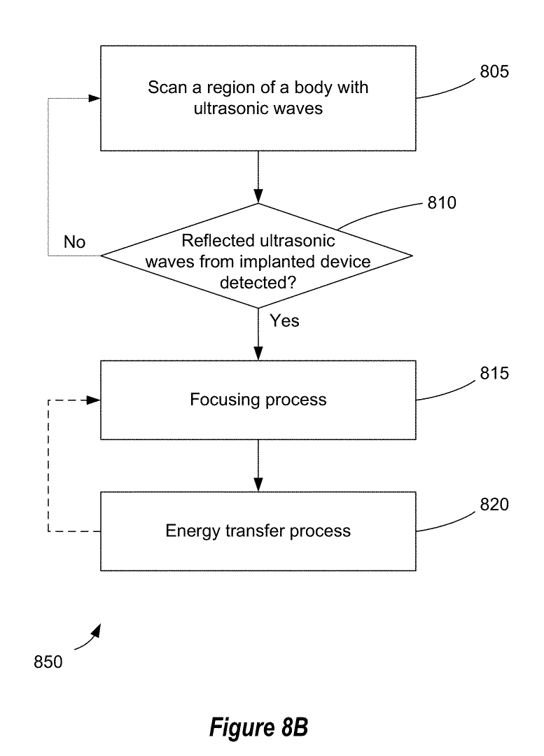

FIG. 8A is a flow diagram that outlines example blocks of a method of locating an implanted device within a human body and providing power to the implanted device. The blocks of method 800 may be performed, for example, by an apparatus 100 as disclosed herein.

According to this example, block 805 involves scanning a region of the body with ultrasonic waves. Block 805 may proceed in a manner similar to that described above with reference to FIG. 7. Block 805 may involve one or more of changing a curvature of a substrate on which the array of PMUTs 105 resides, performing a beam steering process, or changing an orientation of one or more PMUT diaphragms. In some examples, block 805 may involve scanning in a predetermined pattern, such as scanning a spiral pattern, scanning along rows or columns of a predetermined grid, etc.

In this example, block 810 involves determining whether a transmission from an implanted device 315 is detected. In this implementation, the implanted device 315 includes a communication module 705. In some examples, the communication module 705 may be capable of transmitting acoustic waves, such as ultrasonic waves, that may be detected by at least some PMUTs (such as the PMUTs 600b) of the PMUT array 105. Accordingly, in some such examples block 810 may involve determining whether a transmission of acoustic waves, such as ultrasonic waves, from an implanted device is detected.

However, in alternative implementations the communication module 705 may be capable of transmitting other types of signals, such as electromagnetic signals. The apparatus 100 may include a receiver capable of detecting such signals. In some such examples, block 810 may involve determining whether a transmission of electromagnetic signals such as radio frequency signals from an implanted device is detected.

If no transmission from an implanted device is detected, the process may revert to block 805 and the scanning process may be continued. However, if a transmission from an implanted device is detected, the focusing process of block 815 is performed in this example. According to some implementations, block 815 may involve changing a curvature of a substrate on which the array of PMUTs 105 resides, performing a beam steering process and/or changing an orientation of one or more PMUT diaphragms.

In some examples, block 815 may involve evaluating a power level of a transmission to an implanted device. For example, block 815 may involve changing a focus of ultrasonic waves emitted by the PMUT array 105 by changing a curvature of the substrate 305 on which the array of PMUTs 105 resides, performing a beam steering process, and/or changing an orientation of one or more PMUT diaphragms, and evaluating a power level of a transmission to an implanted device according to a current focal area. According to some examples, the process may continue until the current focal area results in a maximum power level of the transmission to the implanted device. According to some implementations, the implanted device may send (e.g., via a communication module of the implanted device) information that indicates, either directly or indirectly, a power level of a transmission sent by the PMUT array 105 and received by the implanted device. In some examples, the implanted device may provide information that indicates, either directly or indirectly, how close the current focal area is to a target location. For example, the communication module may send information indicating whether the current focal area is impinging on a portion of an outer surface of the power receiving module 710, on an entire outer surface of the power receiving module 710 or on no portion of the outer surface of the power receiving module 710.

After the focusing process of block 815, method 800 continues to the energy transfer process of block 820. In some implementations, block 820 may involve increasing the intensity of transmitted ultrasonic waves to a maximum level. In some examples, block 820 may involve adjusting the intensity and/or the frequency of transmitted ultrasonic waves to one or more predetermined levels. The predetermined level(s) may, in some instances, correspond with power transfer information received from the implanted device.

According to some examples, method 800 may revert to the focusing process of block 815 during, or between instances of, the energy transfer process of block 820. For example, method 800 may revert to the focusing process of block 815 at predetermined time intervals. Alternatively, or additionally, method 800 may revert to the focusing process of block 815 upon receiving an indication from the implanted device that a level of power transfer has diminished (e.g., based on the strength of the signal transmitted from the communication module 705) and/or an indication that the current focal area no longer corresponds with a target location, such as a location of the power receiving module 710.

According to some implementations, a control system in communication with the PMUT array (e.g., a control system of a shallow implanted device) may periodically evaluate a power level of the ultrasonic energy received by the deeply-implanted device and may adjust beamforming parameters accordingly. In some such implementations, the deeply-implanted device may be configured to adjust the power level of the ultrasonic energy that the deeply-implanted device is transmitting according to the power level of the ultrasonic energy that the deeply-implanted device is receiving. In some examples, the beamforming parameters may be modified as the person in whom the device is implanted bends or otherwise moves.

FIG. 8B is a flow diagram that outlines example blocks of an alternative method of locating an implanted device within a human body and providing power to the implanted device. The blocks of method 850 may be performed, for example, by an apparatus 100 as disclosed herein. Method 850 may be advantageous for use with an implanted device that lacks a feature such as a communication module 705. Alternatively, method 850 may be advantageous for use with an implanted device that includes a feature such as the communication module 705, but for instances during which the communication module 705 is not responding.

According to this example, block 805 involves scanning a region of the body with ultrasonic waves. Block 805 may proceed in a manner similar to that described above with reference to FIGS. 7 and 8A. Block 805 may involve changing a curvature of a substrate on which the array of PMUTs 105 resides, performing a beam steering process and/or changing an orientation of one or more PMUT diaphragms. In some examples, block 805 may involve scanning in a predetermined pattern, such as scanning a spiral pattern, scanning along rows or columns of a predetermined grid, etc.

In this example, block 810 involves determining whether one or more reflected ultrasonic waves from an implanted device are detected. According to some examples, block 810 may involve determining whether one or more received ultrasonic waves indicate a high impedance contrast, potentially corresponding with a boundary between an implanted device and human tissue. If no reflected ultrasonic waves from an implanted device are detected, the process may revert to block 805 and the scanning process continued.

In the example described above with reference to FIG. 7, the transmissions in the directions T₁₋T₃ did not result in any response from the communication module 705, so the scanning process continued. If this process had taken place according to method 850, block 810 would have involved determining whether one or more reflected ultrasonic waves from an implanted device were detected in response to the transmissions in the directions T₁₋T₃.

If not, the scanning process would have continued: the control system of the apparatus 100 would have caused at least some PMUTs 600a to transmit ultrasonic waves in a direction T₄ at a fourth time. As shown in FIG. 7, the ultrasonic waves that were transmitted in a direction T₄ resulted in the reflected ultrasonic waves R from the implanted device. The reflected ultrasonic waves R may be detected by at least some PMUTs of a PMUT array 105, such as the PMUTs 600b shown in FIG. 7.

After reflected ultrasonic waves from an implanted device are detected, the focusing process of block 815 is performed in this example. According to some implementations, block 815 may involve changing the curvature of the substrate, performing a beam steering process and/or changing an orientation of one or more PMUT diaphragms. In some examples, block 815 may involve a process of detecting one or more features of the implanted device. For example, a control system may be configured to recognize an area of high acoustic impedance contrast between a deeply-implanted device and human tissue.

In some examples, block 815 may involve a process of detecting one or more features on the implanted device via an ultrasonic imaging and pattern recognition process. The pattern may, for example, be a shape of the deeply-implanted device, a pattern of variable acoustic impedance of the deeply-implanted device, etc. In some such examples, block 815 may involve detecting an outline of an outer surface of the power receiving module 710. In some such examples, block 815 may involve detecting a predetermined target shape, such as a fiducial or the rings and/or center circle of a “bullseye” on the outer surface of the power receiving module 710.

In some instances, block 815 (or another process of the method 850) may involve detecting a code or other pattern that corresponds with a particular type of implanted device. The code, pattern and/or shape may correspond with information, such as power transfer information, for the implanted device. In some such examples, an apparatus 100 may refer to a stored data structure that indicates implanted device types and implanted device information, such as power transfer information, the location of a power receiving module on an implanted device, etc.

After the focusing process of block 815, method 850 continues to the energy transfer process of block 820. In some implementations, block 820 may involve increasing the intensity of transmitted ultrasonic waves to a maximum level. In some examples, block 820 may involve adjusting the intensity and/or the frequency of transmitted ultrasonic waves to a predetermined level. The predetermined level may, in some instances, correspond with power transfer information that a control system has determined based on a predetermined shape, code, etc., on the implanted device.

According to some examples, method 850 may revert to the focusing process of block 815 during, or between instances of, the energy transfer process of block 820. For example, method 800 may revert to the focusing process of block 815 at predetermined time intervals.

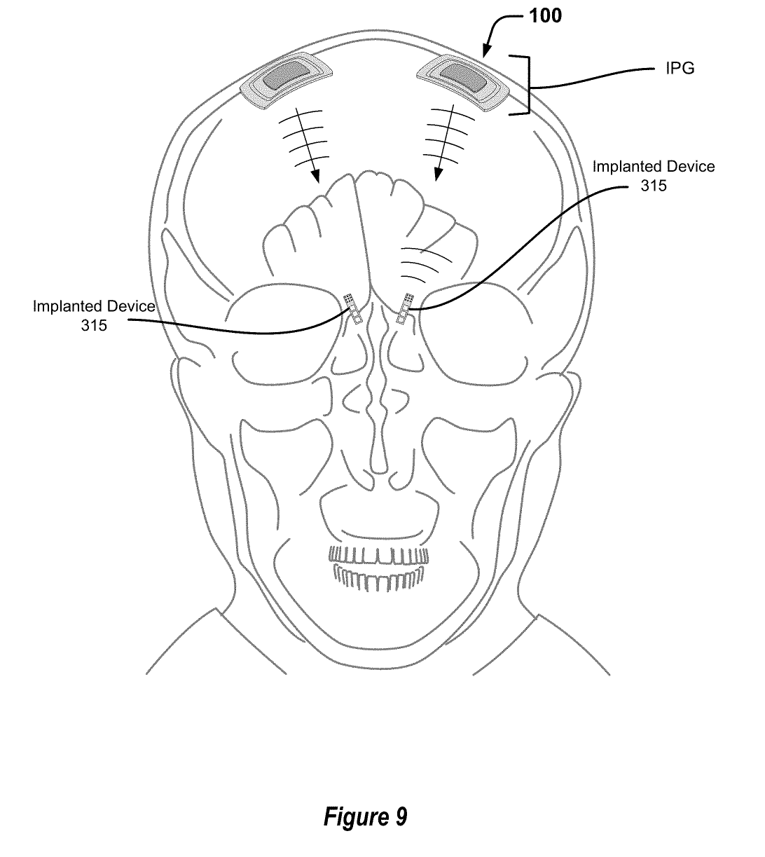

FIG. 9 shows an example of apparatus configured to provide power to implantable devices used for deep brain stimulation (DBS). DBS is a neurosurgical procedure that may be implemented to ameliorate Parkinson's disease, chronic pain, post-traumatic stress disorder and other ailments. In this example, each of the implanted devices 315 includes a neural stimulator. In this example, the apparatus 100, which is also labeled as an implantable pulse generator (IPG) in FIG. 9, is configured to transmit power to the implanted devices 315.

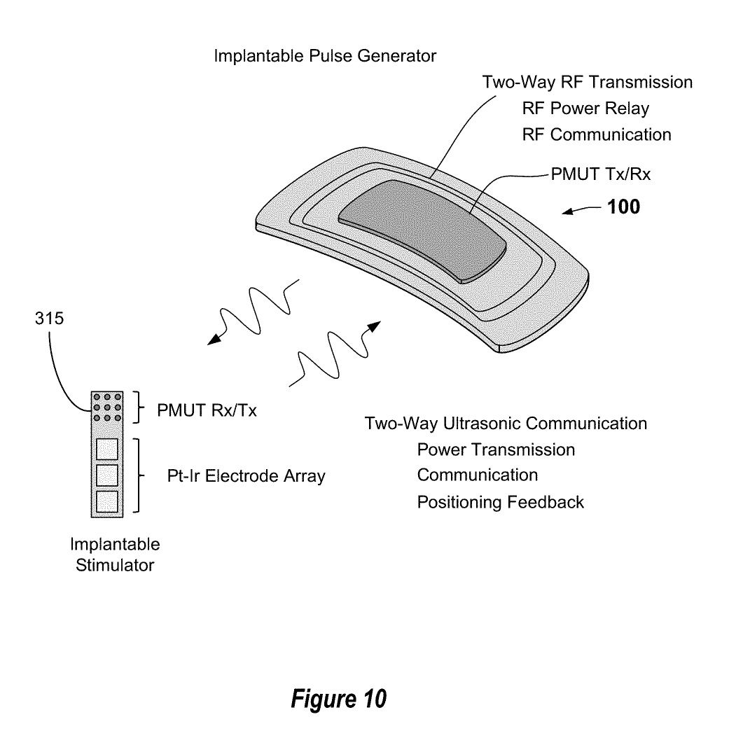

FIG. 10 shows a more detailed example of one of the implantable devices of FIG. 9. According to this example, the deeply-implanted device 315 includes a PMUT array that is configured for receiving and transmitting ultrasonic waves. In some such examples, this PMUT array may provide at least some of the functionality of the communication module 705 and the power receiving module 710 that is described elsewhere herein. In this implementation, the implanted device 315 also includes an electrode array and a control system configured for controlling the electrode array to apply electric signals for DBS. Electrodes of the electrode array are formed of, or at least plated with, a platinum-iridium alloy in this example. Platinum-iridium alloys have excellent biocompatible properties and therefore are suitable for use in implanted devices. However, alternative implementations may include electrodes made of other biocompatible materials.

In the example shown in FIG. 10, the apparatus 100 is configured not only to provide power to the implanted device 315, but also for two-way ultrasonic communication with the implanted device 315. In some such implementations, the implanted device 315 may be configured to provide power transfer information, such as positioning, intensity, frequency and/or focusing information, to the apparatus 100.

According to some implementations, the apparatus 100 may include an electromagnetic transceiver, such as an RF transceiver. The electromagnetic transceiver may be configured for communication with another device, such as a mobile device. In some such implementations, the apparatus 100 may be controlled, at least in part, according to instructions received from another device via the electromagnetic transceiver.

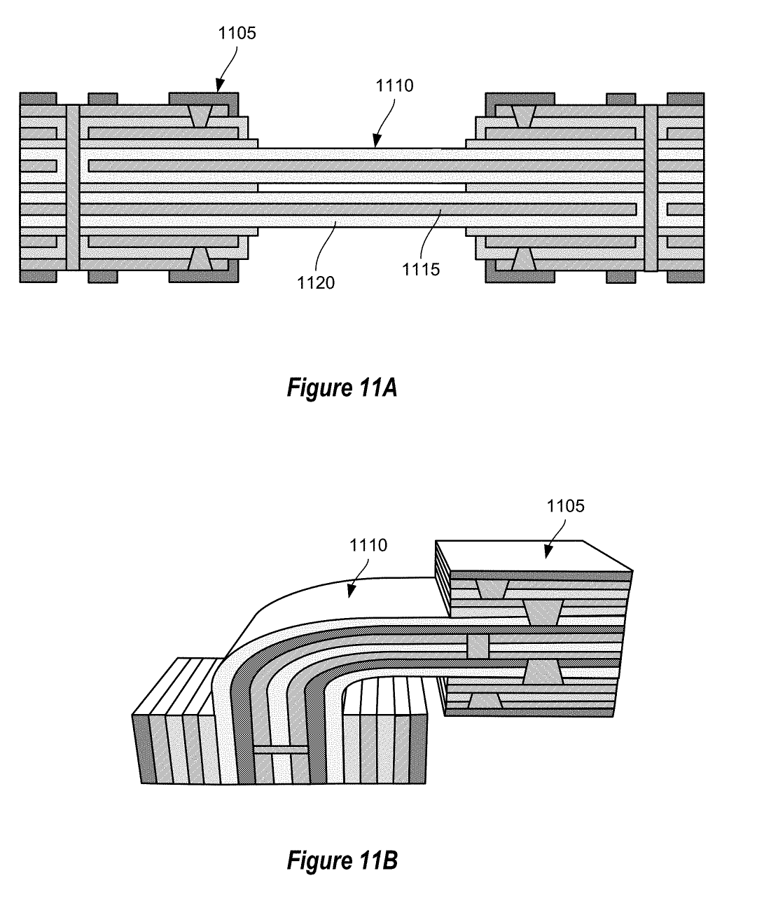

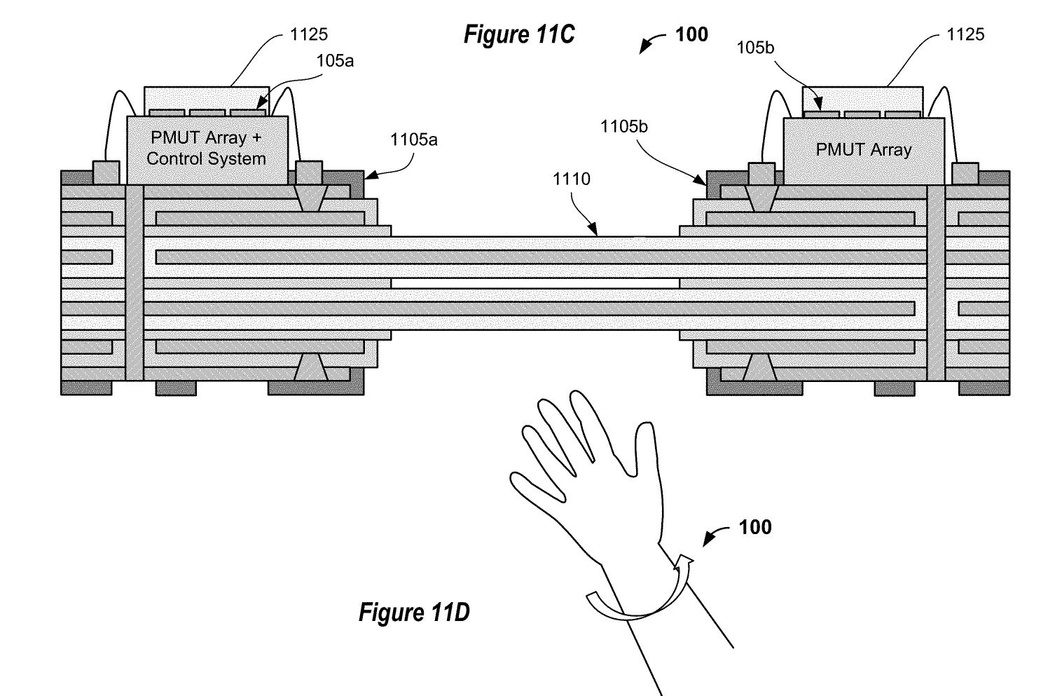

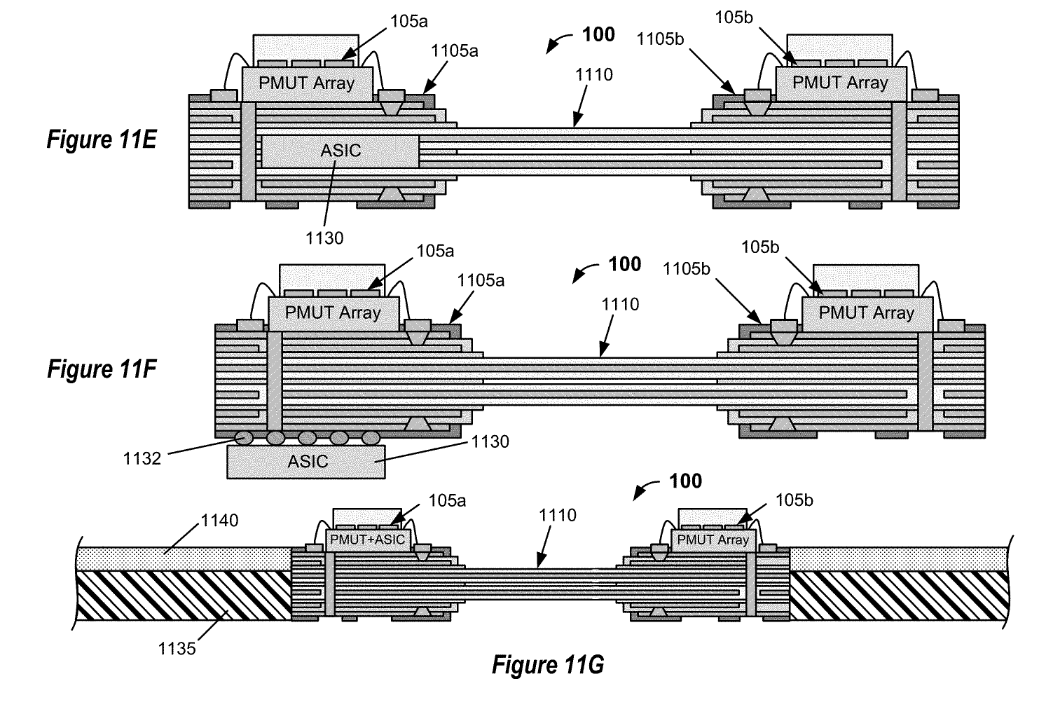

According to some examples, multiple PMUT arrays may be attached to relatively rigid substrates that are connected by relatively flexible routing portions to enable an extended or “super array” with flexible portions and adjustable curvature. FIGS. 11A and 11B show examples of relatively rigid substrates that are connected by relatively flexible routing portions. In these examples, the printed circuit board (PCB) stacks 1105 are connected by the flexible routing portions 1110. Here, the flexible routing portions 1110 include layers of conductive material 1115 separated by dielectric material 1120. The flexible routing portions 1110 may include one or more layers of air or air gaps between conductive or dielectric layers to further increase the flexibility of the flexible routing portions 1110. As shown in FIG. 11B, the flexible routing portions 1110 allow the PCB stacks 1105 to be positioned at adjustable angles, e.g., on or around a curved surface of a person's body.

FIGS. 11C-11G show examples of PMUT arrays that are connected by relatively flexible routing portions. In the example shown in FIG. 11C, the PMUT array 105a resides on the PCB stack 1105a and the PMUT array 105b resides on the PCB stack 1105b. A compliant coupling layer 1125 resides on each of the PMUT array 105a and the PMUT array 105b in this example. The coupling layer 1125 is configured to enhance the coupling of ultrasonic waves into a person's body. The PCB stack 1105a and the PCB stack 1105b are connected by the flexible routing portion 1110, which is configured to conduct electricity between the PCB stack 1105a and the PCB stack 1105b. Other examples may include more than two PCB stacks and more than two PMUT arrays.

In the example shown in FIG. 11C, at least a portion of a control system also resides on the PCB stack 1105a. In some examples the control system, or the portion of the control system, may include a complementary metal-oxide-semiconductor (CMOS) chip or an ASIC. In some implementations, the PMUT array and CMOS circuitry may be co-fabricated on the same (e.g., monolithic) substrate.

Implementations such as those shown in FIG. 11C may, for example, be suitable for providing multiple PMUT arrays 105 in a wearable device. Such implementations have various potential advantages. One advantage is that, as suggested by the arrow that is shown curving around a wrist in FIG. 11D, the flexible routing portion(s) 1110 of the apparatus 100 may be adjusted to conform to at least a portion of a person's body. Accordingly, such implementations have the potential advantage of providing beamforming using multiple PMUT arrays in different locations of a person's body, and potentially positioned at different angles.

FIG. 11E shows an implementation that requires lower-density connections than that of the example shown in FIG. 11C. In FIG. 11E, a PMUT die and the array of PMUTs 105a resides on the PCB stack 1105a, whereas at least a portion of the control system (which is an embedded ASIC 1130 in this example) resides within the PCB stack 1105a.

FIG. 11F shows an implementation that requires lower-density connections than those shown in FIG. 11C or 11E. In FIG. 11F, a PMUT die and the array of PMUTs 105a resides on one side of the PCB stack 1105a, whereas at least a portion of the control system (which is an ASIC 1130 in this example) resides on an opposing side of the PCB stack 1105a. In this example, the ASIC 1130 is connected to conductive material of the PCB stack 1105a via one or more solder bumps 1132.

FIG. 11G shows another example of an implementation like that of FIG. 11C. In this example, the apparatus 100 includes a strap 1135 with a layer of adhesive material 1140 disposed thereon. The adhesive material 1140 may be advantageous for securing the apparatus 100 to a person's body. In some implementations, a portion of the adhesive material 1140 may extend over the PMUT arrays 105a, 105b to serve as a compliant coupling layer for enhancing the coupling of ultrasonic waves to and from a person's body.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that can be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower”, “over” and “under”, and “overlying” and “underlying” are sometimes used for ease of describing the figures, and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

1. An apparatus, comprising:
an array of piezoelectric micromachined ultrasonic transducers (PMUTs); and
a control system configured to communicate with the array of PMUTs, the control system being further configured to:
determine a target location within a human body; and
control the array of PMUTs to focus ultrasonic waves at the target location.

2. The apparatus of claim 1, further comprising a substrate on which at least a portion of the array of PMUTs is disposed, the substrate having a curvature that is configured to focus ultrasonic energy emitted by the PMUTs that are disposed on the substrate.

3. The apparatus of claim 1, wherein one or more PMUTs in the array of PMUTs has a curved surface when in a static position.

4. The apparatus of claim 1, wherein one or more PMUTs in the array of PMUTs includes:
a piezoelectric layer;
a first electrode on a first side of the piezoelectric layer; and
a second electrode on a second side of the piezoelectric layer.

5. The apparatus of claim 4, wherein one or more of the PMUTs does not include a deformable structural layer proximate the first side or the second side of the piezoelectric layer.

6. The apparatus of claim 4, wherein the piezoelectric layer, the first electrode and the second electrode reside on a support structure and wherein at least a portion of a support structure area extends beyond an area of the piezoelectric layer.

7. The apparatus of claim 4, wherein the first electrode is at least one of a center electrode or a ring electrode.

8. The apparatus of claim 4, wherein a first portion of the piezoelectric layer spans a cavity region and a second portion of the piezoelectric layer is mechanically coupled to a support structure adjacent the cavity region, and wherein the second portion of the piezoelectric layer and the support structure combine to produce a mechanical moment on the first portion of the piezoelectric layer when a transmitter excitation signal is applied to one of the first electrode or the second electrode, and wherein the produced mechanical moment results in a transverse deflection of the one or more PMUTs in the array of PMUTs.

9. The apparatus of claim 8, wherein the first electrode and the second electrode span the entire cavity region.

10. The apparatus of claim 8, wherein the one or more PMUTs in the array of PMUTs further includes a deformable structural layer that spans the cavity region.

11. The apparatus of claim 1, wherein controlling the array of PMUTs to focus ultrasonic energy at the target location involves at least one of changing a curvature of a substrate on which the array of PMUTs resides, performing a beam steering process, or changing an orientation of one or more PMUT diaphragms.

12. The apparatus of claim 1, wherein one or more PMUTs of the array of PMUTs is configured to detect received ultrasonic waves and wherein determining the target location is based, at least in part, on received ultrasonic waves that are reflected from or transmitted from the target location.

13. The apparatus of claim 12, wherein the control system is further configured to control the array of PMUTs to scan a region inside the human body with transmitted ultrasonic waves.

14. The apparatus of claim 12, wherein the array of PMUTs includes one or more PMUTs configured to transmit ultrasonic waves, the one or more PMUTs configured to transmit ultrasonic waves including a piezoelectric material having at least one of a higher piezoelectric coefficient, a higher dielectric constant and a smaller thickness relative to the piezoelectric material of the one or more PMUTs configured to detect received ultrasonic waves.

15. The apparatus of claim 1, wherein the control system is further configured to control at least one of a power level or a focal area of at least a portion of the array of PMUTs according to one or more signals received from a device implanted within the human body.

16. The apparatus of claim 1, wherein the target location corresponds with at least a portion of a device implanted within the human body.

17. The apparatus of claim 16, wherein the target location corresponds with a second array of PMUTs of the device implanted within the human body.

18. The apparatus of claim 16, wherein the control system is configured to control the array of PMUTs for ultrasonic energy transmission to the device implanted within the human body.

19. The apparatus of claim 1, wherein one or more PMUTs in the array of PMUTs includes at least one edge electrode that is configured to orient a PMUT diaphragm in the array of PMUTs towards the target location.

20. A method of controlling an array of piezoelectric micromachined ultrasonic transducers (PMUTs), the method comprising:
determining a target location within a human body based, at least in part, on received ultrasonic waves that are reflected from or transmitted from the target location, the received ultrasonic waves being received by one or more PMUTs of the array of PMUTs configured for detecting received ultrasonic waves; and
controlling the array of PMUTs to focus ultrasonic waves at the target location.

21. The method of claim 20, further comprising controlling the array of PMUTs to scan a region inside the human body with transmitted ultrasonic waves.

22. The method of claim 20, further comprising controlling the array of PMUTs to transmit ultrasonic energy to a device implanted within the human body.

23. The method of claim 20, further comprising controlling at least one of a power level or a focal area of at least a portion of the array of PMUTs according to one or more signals received from a device implanted within the human body.

24. A non-transitory medium having software stored thereon, the software including instructions for causing a processor to:
determine a target location within a human body based, at least in part, on received ultrasonic waves that are reflected from or transmitted from the target location, the received ultrasonic waves being received by one or more piezoelectric micromachined ultrasonic transducers (PMUTs) of an array of PMUTs configured for detecting received ultrasonic waves; and
control the array of PMUTs to focus ultrasonic waves at the target location.

25. The non-transitory medium of claim 24, wherein the software further includes instructions for causing a processor to: control the array of PMUTs to scan a region inside the human body with transmitted ultrasonic waves.

26. The non-transitory medium of claim 24, wherein the software further includes instructions for causing a processor to: control the array of PMUTs to transmit ultrasonic energy to a device implanted within the human body.

27. The non-transitory medium of claim 24, wherein the software further includes instructions for causing a processor to: control at least one of a power level or a focal area of at least a portion of the array of PMUTs according to one or more signals received from a device implanted within the human body.

28. An apparatus, comprising:
an array of piezoelectric micromachined ultrasonic transducers (PMUTs); and
control means for communication with the array of PMUTs, the control means including means for:
determining a target location within a human body; and
controlling the array of PMUTs to focus ultrasonic waves at the target location.

29. The apparatus of claim 28, wherein the control means includes means for controlling the array of PMUTs to scan a region inside the human body with transmitted ultrasonic waves.

30. The apparatus of claim 28, wherein the control means includes means for controlling the array of PMUTs to transmit ultrasonic energy to a device implanted within the human body.
